(19) United States
(12) Patent Application Publication
(10) Pub. No.: US 2023/0076975 A1
(43) Pub. Date: Mar. 9, 2023

(54) PEPTIDE AND PROTEIN C-TERMINUS LABELING

(71) Applicant: BOARD OF REGENTS, THE UNIVERSITY OF TEXAS SYSTEM, Austin, TX (US)

(72) Inventors: Eric V. ANSLYN, Austin, TX (US); Edward MARCOTTE, Austin, TX (US); Cecil J. HOWARD, II, Austin, TX (US); Jagannath SWAMINATHAN, Austin, TX (US); Angela M. BARDO, Austin, TX (US); Caroline M. HINSON, Austin, TX (US); Brendan FLOYD, Austin, TX (US); Le ZHANG, Austin, TX (US)

(21) Appl. No.: 17/820,646

(22) Filed: Aug. 18, 2022

Related U.S. Application Data
(63) Continuation of application No. PCT/US2021/018535, filed on Feb. 18, 2021.
(60) Provisional application No. 62/978,035, filed on Feb. 18, 2020.

(51) Int. Cl. C07K 1/13; C12N 11/06; G01N 33/58

(52) U.S. Cl. CPC C07K 1/13; C12N 11/06; G01N 33/58

(57) ABSTRACT

Described herein are methods for selectively cleaving the C-terminal amino acid of a peptide or protein. The methods described herein may be applicable for, for example, single-molecule peptide or protein sequencing.

This application is a continuation of International Application No. PCT/US2021/018535, filed Feb. 18, 2021, which claims the benefit of priority to U.S. Provisional Application Ser. No. 62/978,035, filed on Feb. 18, 2020, the entire contents of which are hereby incorporated by reference.

### STATEMENT AS TO FEDERALLY SPONSORED RESEARCH

This invention was made with government support under Grant no. R35 GM122480 awarded by the National Institutes of Health. The government has certain rights in the invention.

### BACKGROUND

Synthetic techniques have been developed for selective and efficient labeling of reactive amino acid side chains on peptide molecules. Methodologies for discriminating the N-terminal amino acid from internal amino acid residues (e.g., lysine) has also been explored. However, methodologies for discriminately attaching a chemical handle to the C-terminus of a peptide or protein are not amenable to generalized procedures. This is intrinsically challenging since, for example, (i) the reactivity of the acidic amino acid residues (e.g., aspartic acid and glutamic acid) are similar and (ii) the acidic side chains of the residues are about 50 times more abundant than the C-terminal acidic moiety. Overcoming the challenge of ligating proteins and peptides to a fixed handle via the C-terminus without any bias caused by the identity of the terminal amino acid is needed, such as, in proteomics research.

### SUMMARY

Described herein are compositions and methods (e.g., chemical and enzymatic) to selectively modify the C-terminal carboxylic acid of proteins and peptides. Ligation methods include the use of, for example, oxazolone-based chemistry, photoredox chemistry, carboxypeptidases (e.g., carboxypeptidase Y), and peptiligases (e.g., Omniligase). In another aspect, described herein are compositions comprising handles for selectively reacting peptide C-termini, hereinafter referred to as C-terminal coupling reagents. The methods and compositions described herein can provide a heterogeneous population of peptides, all containing a constant C-terminal coupling reagent optionally configured for any number of applications, such as, for example, protein and peptide (i) surface immobilization, (ii) multiplexing (e.g., via chemical barcodes), (iii) enrichment, (iv) fluorosequencing (e.g., single molecule protein sequencing), and (v) nanopore translocation and sequencing.

For example, FIG. 1A exemplifies the discriminatory capability of the compounds and methods described herein. The C-terminal carboxylic acid residue of peptides, proteins, or combinations thereof can be discriminated between internal amino acids comprising carboxylic acid residues (e.g., glutamic acid and aspartic acid) using enzymatic methods, chemical methods, or a combination thereof. The methods and compositions described herein can produce proteins, peptides, or a combination thereof modified via the C-terminal amino acid residue (e.g., coupled to a C-terminal coupling reagent. In some embodiments, the C-terminal carboxylic acid residues of peptides, proteins, or combinations thereof can be discriminated from internal amino acid residues containing carboxylic acid amino acid residues using compositions and methods described herein. Depending on the composition of the handle (e.g., FIG. 1B), these proteins, peptides, or combinations thereof can be manipulated as described herein to accomplish a variety of proteomics applications, such as, for example, fluorosequencing (FIG. 2). In some embodiments, the methods and compositions described herein are applicable for single-molecule fluorosequencing of proteins, peptides, or combinations thereof. Selectively labeling the C-terminus of a protein or peptide (e.g., coupling a C-terminal coupling reagent to the protein or peptide C-terminus) can provide, for example, a handle for coupling to a surface, a reference to determine the location of a peptide or protein, and a barcode to determine the identity of the peptide or protein.

In certain aspects, described herein is a method for processing a peptide or a protein comprising a C-terminus, which comprises a first carboxylic acid moiety, and an internal amino acid residue, which comprises a second carboxylic acid moiety, the method comprising coupling said first carboxylic acid moiety with a reactive agent (e.g., a C-terminal coupling reagent) preferentially over said second carboxylic acid moiety. In some embodiments, coupling said first carboxylic acid moiety with said reactive agent is at least 50% more preferential than coupling said second carboxylic acid moiety with said reactive agent. In some embodiments, coupling said first carboxylic acid moiety with said reactive agent is at least 75% more preferential than coupling said second carboxylic acid moiety with said reactive agent. In some embodiments, coupling said first carboxylic acid moiety with said reactive agent is at least 90% more preferential than coupling said second carboxylic acid moiety with said reactive agent. In some embodiments, coupling said first carboxylic acid moiety with said reactive agent is at least 95% more preferential than coupling said second carboxylic acid moiety with said reactive agent. In some embodiments, coupling said first carboxylic acid moiety with said reactive agent is at least 98% more preferential than coupling said second carboxylic acid moiety with said reactive agent. In some embodiments, coupling said first carboxylic acid moiety with said reactive agent is at least 99% more preferential than coupling said second carboxylic acid moiety with said reactive agent. In some embodiments, coupling said first carboxylic acid moiety with said reactive agent is at least 99.99% more preferential than coupling said second carboxylic acid moiety with said reactive agent. In some embodiments, the peptide or the protein is immobilized (e.g., to a substrate such as a glass slide, a nanoparticle, or a microparticle).

In certain aspects, described herein is a method for processing a peptide or a protein comprising a C-terminus, which comprises a first carboxylic acid moiety (e.g., the C-terminal amino acid carboxyl and not a C-terminal side chain), and an internal amino acid residue, which comprises a second carboxylic acid moiety, the method comprising coupling said first carboxylic acid moiety of said immobilized peptide or said protein with a reactive agent preferentially over said second carboxylic acid moiety of said peptide or protein. In some embodiments, the peptide or the protein is immobilized.

In certain aspects, described herein is a method for processing a peptide or protein comprising a C-terminus, which comprises a first carboxylic acid moiety, and an internal amino acid residue, which comprises a second carboxylic acid moiety, the method comprising coupling said first carboxylic acid moiety of said peptide or protein with a reactive agent preferentially over said second carboxylic acid moiety of said peptide or protein, wherein said reactive reagent comprises a functionalization moiety, an enrichment moiety, or a combination thereof.

In certain aspects, described herein is a method for processing a peptide or a protein comprising a C-terminus, which comprises a first carboxylic acid moiety, and an internal amino acid residue, which comprises a second carboxylic acid moiety, the method comprising coupling a reactive agent (e.g., a C-terminal coupling reagent) to said first carboxylic acid moiety in absence of coupling said reactive agent to said second carboxylic acid moiety. In some embodiments, said peptide or protein comprises at least two internal amino acid residues, wherein at least one of said at least two internal amino acid residues comprises said second carboxylic acid moiety. In some embodiments, said peptide or protein comprises at least twenty internal amino acid residues, wherein at least one of said at least twenty internal amino acid residues comprises a second carboxylic acid moiety.

In some embodiments, said reactive agent comprises a label. In some embodiments, said label comprises an optical label (e.g. fluorophore), a nucleic acid molecule (e.g., DNA, RNA, PNA), an ionizable molecule (e.g., a bromine, an amine, a phosphate), a polyethylene spacer, a polyarginine peptide, or any combination thereof. In some embodiments, said nucleic acid molecule comprises a nucleic acid barcode.

In some embodiments, said reactive agent comprises a nucleophile or an electrophile. In some embodiments, said nucleophile comprises an amine, an alcohol, a sulfide, a cyanate, a thiocyanate, a negatively charged species, or any combination thereof. In some embodiments, said electrophile comprises a Michael acceptor, an alkene, a diene, an acrylamide, an N-(prop-2-yn-1-yl)methylacrylamide, an isocyanate, an isothiocyanate, a conformationally constrained moiety (e.g., an oxirane, an α, β-unsaturated carbonyl), a vinyl sulfone, or any combination thereof.

In some embodiments, said reactive agent comprises a functionalization moiety, an enrichment moiety, or a combination thereof. In some embodiments, said functionalization moiety comprises an alkyne, an azide, a fluorophore, biotin, a nucleic acid molecule (e.g., RNA, DNA, PNA), an amino acid, a peptide, a solid support bead or resin, or any combination thereof. In some embodiments, said enrichment moiety comprises an alkyne, an azide, a fluorophore, biotin, a nucleic acid molecule (e.g., RNA, DNA, PNA), an amino acid, a peptide, a solid support bead or resin, or any combination thereof.

In some embodiments, the method further comprises treating said peptide or protein with at least one chemical, at least one enzyme, or a combination thereof. In some embodiments, said at least one chemical is a photocatalyst. In some embodiments, said photocatalyst is lumiflavin. In some embodiments, said at least one chemical reacts with said peptide or protein to form an oxazolone intermediate of said peptide or protein. In some embodiments, said at least one chemical comprises acetic anhydride, a hydroxybenzotriazole (HOBT), a hydroxyazabenzotriazole (HOAT), 2-nitro-5-thiobenzoic acid (NTCB), or a combination thereof. In some embodiments, said at least one enzyme is an endopeptidase, an exopeptidase, a carboxypeptidase, an amidase, a hydrolase, a proteinase, a peptiligase, or any combination thereof. In some embodiments, said peptiligase is an Omniligase. In some embodiments, said peptiligase is an enzyme that catalyzes peptide coupling in water. In some embodiments, said carboxypeptidase is a Carboxypeptidase Y. In some embodiments, said proteinase is a thermolysin.

In some embodiments, the method comprises cleaving a plurality of peptides or proteins, wherein said plurality of peptides or proteins comprises said peptide or protein. In some embodiments, said reactive agent does not substantially couple to (i) said at least one internal amino acid residue and (ii) an N-terminal amino acid residue of said peptide or protein. In some embodiments, said reactive agent does not substantially couple to any internal amino acid residue of said peptide or protein.

In some embodiments, said at least one internal amino acid residue is a natural amino acid. In some embodiments, said at least one said internal amino acid residue comprises a functional group selected from the group consisting of amines, carboxylic acids, indoles, alcohols, thiols, thioethers, phenols, amides, guanidinium, and imidazoles. In some embodiments, said at least one said internal amino acid residue comprises a functional group selected from the group consisting of amines, carboxylic acids, and thiols. In some embodiments, said at least one said internal amino acid residue is an unnatural amino acid.

In some embodiments, said at least one internal amino acid residue, said N-terminal amino acid residue of said peptide or protein, or a combination thereof is modified prior to coupling said reactive agent to said first carboxylic acid moiety. In some embodiments, said at least one internal amino acid residue, said N-terminal amino acid residue of said peptide or protein, or a combination thereof is modified subsequent to coupling said reactive agent to said first carboxylic acid moiety. In some embodiments, said peptide or protein is reversibly modified.

In some embodiments, said at least one internal amino acid residue is selected from the group consisting of cysteine, lysine, tyrosine, tryptophan, serine, histidine, threonine, and arginine, phosphorylated amino acids, post-translationally modified amino acids, or any combination thereof. In some embodiments, said at least one internal amino acid residue is selected from the group consisting of cysteine and lysine. In some embodiments, said at least one internal amino acid residue is coupled to at least one label. In some embodiments, each internal amino acid of said plurality of internal amino acid residues is coupled to said at least one label. In some embodiments, said at least one label corresponds to a different label for each internal amino acid type.

In some embodiments, said at least one label is an optical label. In some embodiments, said optical label is a fluorophore.

In some embodiments, the method further comprises producing a labeled peptide or protein for surface immobilization, sample multiplexing, sample enrichment, sequencing, target identification, mass spectrometry, or any combination thereof. In some embodiments, said sequencing is single-molecule sequencing, nanopore sequencing, fluorosequencing, or a combination thereof.

In some embodiments, the method further comprises isolating said peptide or protein from a biological sample. In some embodiments, said biological sample is derived from tissue, blood, urine, saliva, lymphatic fluid, or any combination thereof. In some embodiments, said peptide or protein is a recombinant or a synthetic peptide or protein.

In some embodiments, the method further comprises digesting said peptide or protein. In some embodiments, the method further comprises (i) isolating said peptide or protein, (ii) immobilizing said peptide or protein to a solid support, (iii) labeling at least one internal amino acid residue, and (iv) releasing said peptide or protein from said solid support. In some embodiments, said immobilizing said peptide or protein comprises coupling a N-terminal amino acid residue of said peptide or protein to a capture moiety coupled to said solid support. In some embodiments, said capture moiety comprises an aldehyde. In some embodiments, said capture moiety comprises pyridine carboxaldehyde or an analog thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A & 1B schematic of (A) C-terminal carboxylic acid ligation for ligand coupling and (B) C-terminal coupling reagent design.

FIG. 2 illustration of the principle of fluorosequencing technology utilizing C-terminal ligation.

FIG. 3 depicts an example of a chemical method comprising oxazolone chemistry for labeling the C-terminal carboxylic acid with a C-terminal coupling reagent.

FIGS. 4A & 4B depicts MS spectral evidence of labeling peptide's terminal carboxylate with Azide handle. Peptide with sequence H₂N-ELYAEKVATR-OH (SEQ ID NO: 22) is conjugated to the nucleophilic handle (H₂N-PEG4-Azide). A 12 min LC/MS separation of the product was performed (FIG. 4A) and the MS1 spectra (m/z-716.7 with +2 charge) indicates the desired product (FIG. 4B).

FIG. 5A-H shows a reaction scheme of photoredox catalyzed conjugation of the C-terminus of angiotensin. (Asp-Arg-Val-Tyr-Ile-His-Pro=SEQ ID NO: 23) FIG. 5B and FIG. 5C shows the Extracted Ion Chromatogram for the mass-ranges corresponding to 523-524 (5B, angiotensin—eluting with a peak at 5.3 mins) and 594-595 (5C, angiotensin C-terminal adduct e) on the 12-minute LC separation. FIGS. 5D-5H represent high resolution images for FIGS. 5B and 5C.

FIG. 6 shows an example of a C-terminal coupling reagent comprising (a) an amine or Michael acceptor for coupling to a peptide C-terminal carboxylic acid residue, (b) a barcoded nucleic acid oligomer for detection by hybridization and (c) an alkyne residue for click chemistry immobilization with an alkyne functionalized surface.

FIG. 7 illustrates a schematic of multiplexing peptides from different samples for identification and quantification by fluorosequencing technology.

FIG. 8 provides a photograph of a benchtop setup for a photoredox C-terminal labeling assay.

FIG. 9A provides a scheme for a photoredox C-terminal labeling reaction.

FIG. 9B provides liquid chromatography-mass spectrometry (LCMS) results from a photoredox C-terminal labeling assay of Angiotensin II.

FIG. 9C provides a mass spectrum of norbornenone labeled Angiotensin II.

FIG. 10A summarizes C-terminal labeling efficiencies of trypsinized bovine serum albumin (BSA), human protein isolate, and yeast protein isolates with norbornenone through a photoredox coupling assay.

FIG. 10B summarizes C-terminal labeling efficiencies of GluC and trypsin digested, bovine serum albumin (BSA), human protein isolate, and yeast protein isolates with norbornenone through a photoredox coupling assay.

FIG. 11 summarizes C-terminal labeling efficiencies for peptides terminating in a variety of amino acids.

FIG. 12 panel A provides a peptide fluorosequencing scheme that comprises C-terminal and selective amino acid side chain labeling.

FIG. 12 panel B provides a fluorescence image of a plurality of substrate-immobilized, fluorescently labeled peptides.

FIG. 12 panel C provides peptide counts from the assay outlined in FIG. 12 panel A with Angiotensin, a peptide comprising the sequence AK*AGANY{PRA}R—ONH₂ (SEQ ID NO: 24), and peptide-free water.

FIG. 13 provides a table of variable C-terminal labeling efficiencies for peptides comprising different C-terminal amino acid types.

### DETAILED DESCRIPTION

Selectively reacting a C-terminal carboxylic acid of a peptide or protein is not trivial because of, for example, the chemical similarity between the C-terminal carboxylic acid and amino acid residues comprising carboxylic acid moieties (e.g., glutamate and aspartate) of peptides and proteins. The ability to selectively target the C-terminal carboxyl has wide extensive potential in the field of proteomics. Adapting C-terminal labeling with the design of functionalized nucleophilic handles provides utility for a number of methods in single molecule protein sequencing, mass spectrometry, peptide purification, and nanopore technologies. In an aspect, described herein are, for example, (a) methods for selectively reacting agents (e.g., C-terminal coupling reagents) with the C-terminal amino acid of a peptide or protein (b) compositions and agents (e.g., C-terminal coupling reagents) that can selectively react with the C-terminal amino acid of a peptide or protein, and (c) applications and methods for a number of proteomic technologies using C-terminally selective agents described herein, such as, for example, single molecule protein sequencing.

### Terms and Definitions

As used herein, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The term “substantially” or “substantial” as used herein generally refers to at least about 60% or 60%, about 70% or 70%, or about or at 75%, 80%, 85%, 90%, 95%, 96%, 97%, 98%, 99% or higher relative to a reference such as, for example, the original composition or state of an entity. Thus, an agent that does not “substantially” couple to an internal amino acid indicates that at least about 60% or 60%, about 70% or 70%, or about or at 75%, 80%, 85%, 90%, 95%, 96%, 97%, 98%, 99%, or higher amounts of the agent have not reacted with the internal amino acid.

The term “selective” or “selectively”, as used herein, generally refers to a preference of at least about 50% or 50%, about 60% or 60%, about 70% or 70%, or about or at 75%, 80%, 85%, 90%, 95%, 96%, 97%, 98%, 99%, or 100% for one composition than another composition. For example, a reaction that is “selective” for a C-terminal amino acid of a peptide or protein has about a 50% or 50%, about 60% or 60%, about 70% or 70%, or about or at 75%, 80%, 85%, 90%, 95%, 96%, 97%, 98%, 99%, or 100% preference to react with the C-terminal amino acid than another group of the peptide or protein, such as, for example, an internal amino acid of the peptide or protein.

As used herein, the term “amino acid” in general refers to organic compounds that contain at least one amino group, NH₂, which may be present in its ionized form, —NH₃⁺, and one carboxyl group, —COOH, which may be present in its ionized form, —COO⁻, where the carboxylic acids are deprotonated at neutral pH, having the formula of ⁺NH₃CHRCOO⁻. An amino acid and thus a peptide has an N (amino)-terminal residue region and a C (carboxy)-terminal residue region. Types of amino acids may include at least 20 that are considered “natural” as they comprise the majority of biological proteins in mammals and include amino acid, such as, for example, lysine, cysteine, tyrosine, threonine, etc. Amino acids may also be grouped based upon their side chains, such as those with a carboxylic acid groups (at neutral pH), including aspartic acid or aspartate (Asp; D) and glutamic acid or glutamate (Glu; E); and basic amino acids (at neutral pH), including lysine (Lys; L), arginine (Arg; N), and histidine (His; H).

As used herein, the term “terminal” is referred to as singular terminus and plural termini. A “N-terminal amino acid residue” may refer to an amino acid residue at the end of a peptide or protein that has a free NH₂ or NH₃. A “C-terminal amino acid residue” may refer to an amino acid residue at the end of a peptide or protein that has a free COOH or COO⁻.

As used herein, the term “side chains”, “residue”, or “R” refers to groups attached to the α-carbon (the carbon that couples the amine and carboxylic acid groups of an amino acid) that render each type of amino acid (e.g., natural amino acid). R groups have a variety of shapes, sizes, charges, and reactivities, such as, for example, charged polar side chains (e.g., positively or negatively charged, such as, for example, lysine (+), arginine (+), histidine (+), aspartate (−), and glutamate (−)); amino acids can also be basic (e.g., lysine) or acidic (e.g., glutamic acid); uncharged polar side chains may comprise groups, such as, for example, hydroxyl, amide, or thiol groups (e.g., cysteine), which may be a chemically reactive side chain (e.g., a thiol group that can form bonds with another cysteine, serine (Ser) and threonine (Thr)); asparagine (Asn), glutamine (Gin), and tyrosine (Tyr); non-polar hydrophobic amino acid side chains (e.g., glycine, alanine, valine, leucine, and isoleucine) having aliphatic hydrocarbon side chains ranging in size from a methyl group (e.g., alanine) to isomeric butyl groups (e.g., leucine and isoleucine); methionine (Met) has a thiol ether side chain; proline (Pro) has a cyclic pyrrolidine side group. Phenylalanine (with its phenyl moiety) (Phe) and tryptophan (Trp) (with its indole group) contain aromatic side chains, which are characterized by bulk as well as lack of polarity.

Amino acids can be referred to by a name, 3-letter code, or 1-letter code, for example, Cysteine, Cys, C; Lysine, Lys, K; Tryptophan, Trp, W, respectively.

“Unnatural” amino acids are those not naturally encoded or found in the genetic code nor produced via de novo metabolic pathways in mammals and plants. They can be synthesized by adding side chains not normally found or rarely found on amino acids in nature. Examples may include: β-amino acids (e.g., β-alanine), homo-amino acids (e.g., homoserine), proline derivatives (e.g., cis-4-Hydroxy-D-proline), 3-substituted alanine derivatives (e.g., 3,3-diphenyl-D-alanine), glycine derivatives (e.g., sarcosine), ring-substituted phenylalanine and tyrosine derivatives (e.g., 4-chloro-L-phenylalanine and 3-chloro-L-tyrosine, respectively), linear core amino acids (e.g., 4-amino-3-hydroxybutyric acid), and N-methyl amino acids (e.g., L-abrine).

As used herein, β amino acids, which have their amino group bonded to the β carbon rather than the α-carbon as in the 20 standard biological amino acids, are unnatural amino acids. The only common naturally occurring β amino acid is β-alanine.

As used herein, the terms “amino acid sequence”, “peptide”, “peptide sequence”, “polypeptide”, “oligopeptide”, “polypeptide sequence”, and “oligopeptide sequence” as used herein refer to at least two amino acids or amino acid analogs that are covalently linked by a peptide (amide) bond or an analog of a peptide bond. The term peptide includes oligomers and polymers of amino acids or amino acid analogs. The term peptide also includes molecules that may be referred to as oligopeptides, which may contain from about two (2) to about twenty (20) amino acids. The term peptide may include molecules that are commonly referred to as polypeptides, which generally contain more than twenty (20) amino acids. The term peptide also includes molecules that are commonly referred to as proteins, which may contain at least about twenty (20) amino acids and a set of defined structural features (e.g., a set of secondary, tertiary, and quaternary structures). The amino acids of the peptide may be L-amino acids or D-amino acids. A peptide, polypeptide, or protein may be synthetic, recombinant, or naturally occurring. A synthetic peptide is a peptide that is produced by artificial means in vitro.

As used herein, the term “fluorescence” refers to the emission of visible light by a substance that has absorbed light of a different wavelength. Fluorescence may provide a non-destructive way of tracking and/or analyzing biological molecules based on the fluorescent emission at a specific wavelength. Proteins (including antibodies), peptides, nucleic acid, oligonucleotides (including single stranded and double stranded primers) may be “labeled” with a variety of extrinsic fluorescent molecules referred to as fluorophores.

As used herein, sequencing of peptides “at the single molecule level” refers to amino acid sequence information obtained from individual (i.e., single) peptide molecules, which can be in a mixture of diverse peptide molecules. It is not necessary that the present invention be limited to methods where the amino acid sequence information obtained from an individual peptide molecule is the complete or contiguous amino acid sequence of an individual peptide molecule. It may be sufficient that only partial amino acid sequence information is obtained, allowing for identification of the peptide or protein. Partial amino acid sequence information, including for example, the pattern of a specific amino acid residue (i.e., lysine) within individual peptide molecules, may be sufficient to uniquely identify an individual peptide molecule. For example, a pattern of amino acids such as, for instance, X-X-X-Lys-X-X-X-X-Lys-X-Lys (SEQ ID NO: 25), which indicates the distribution of lysine molecules within an individual peptide molecule, may be searched against a known proteome of a given organism to identify the individual peptide molecule. It is not intended that sequencing of peptides at the single molecule level be limited to identifying the pattern of lysine residues in an individual peptide molecule; sequence information for any amino acid residue (including multiple amino acid residues) may be used to identify individual peptide molecules in a mixture of diverse peptide molecules.

As used herein, “single molecule sensitivity” refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). This may include the ability to simultaneously record the fluorescent intensity of multiple individual (i.e., single) peptide molecules distributed across the glass surface. Optical devices are commercially available that can be applied in this manner. For example, a conventional microscope equipped with total internal reflection illumination and an intensified charge-couple device (CCD) detector is available (see Braslaysky et al., 2003). Imaging with a high sensitivity CCD camera allows the instrument to simultaneously record the fluorescent intensity of multiple individual (i.e., single) peptide molecules distributed across a surface. Image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

The term “single-cell proteomics”, as used herein, refers to the study of the proteome of a cell. The proteome may be of a single cell. The proteome may be of a cluster of cells. The cluster of cells may be at least two cells. The cluster of cells may be 2, 5, 10, 20, 30, 40, 50, 60, 70, 80, 90, 100, or more cells. The cluster of cells may be from 2 to 10 cells. In some embodiments, the proteome of a single cell comprises proteins, peptides, or a combination thereof. In some embodiments, studying the proteome comprises determining the amino acid sequence for at least one peptide, protein, or combination thereof. In some embodiments, the amino acid sequence is determined by sequencing peptides, proteins, or a combination thereof. The cells may be eukaryotic, prokaryotic, or archaean.

The term “support”, as used herein, refers to as a solid or semi-solid support. In some embodiments, the support is a bead or a resin.

The term “barcode” or “barcode sequence” as used herein, refers to a molecule that can be identified to distinguish a probe, a peptide, a protein, or any combination thereof from another probe, peptide, protein, or any combination thereof. In general, a barcode or barcode sequence labels a molecule or provides a molecule with an identity. The barcode can be an artificial molecule or a naturally occurring molecule. In some embodiments, at least a portion of the barcodes in a population of barcodes comprise barcodes that are different from another barcode in the population of barcodes. In some embodiments, at least about 10%, 20%, 30%, 40%, 50%, 60%, 70%, 80%, 90%, 95%, 99%, or more of the barcodes are different. The diversity of different barcodes in a population of barcodes can be randomly generated or non-randomly generated.

The term “nucleic acid barcode sequence”, as used herein, refers to a molecule with a particular sequence of nucleic acid. Generally, a nucleic acid barcode sequence can include one or more nucleotide sequences that can be used to identify one or more particular nucleic acids. The nucleic acid barcode sequence can be an artificial sequence or can be a naturally occurring sequence. A nucleic acid barcode sequence can comprise at least about 1, 2, 3, 4, 5, 6, 7, 8, 9, 10, 11, 12, 13, 14, 15, 16, 17, 18, 19, 20 or more consecutive nucleotides. In some embodiments, a nucleic acid barcode sequence comprises at least about 10, 20, 30, 40, 50, 60, 70, 80, 90, 100 or more consecutive nucleotides. In some embodiments, at least a portion of the nucleic acid barcode sequences in a population of nucleic acids comprising barcodes is different. In some embodiments, at least about 10%, 20%, 30%, 40%, 50%, 60%, 70%, 80%, 90%, 95%, 99%, or more of the nucleic acid barcode sequences are different. The diversity of different nucleic acid barcode sequences in a population of nucleic acids comprising nucleic acid barcode sequences can be randomly generated or non-randomly generated.

The term “nucleic acid” as used herein generally refers to a polymeric form of nucleotides of any length, either ribonucleotides (RNA), deoxyribonucleotides (DNA) or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus, the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. The nucleic acid molecule may be a DNA molecule. The nucleic acid molecule may be an RNA molecule.

The sequencing reactions may comprise, for example, capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. The single molecule sequencing may provide single molecule resolution. Sequential nucleotide flow sequencing may comprise pyrosequencing, pH-mediated sequencing, semiconductor sequencing or a combination thereof. Conducting one or more sequencing reactions may comprise whole genome sequencing or exome sequencing. The hybridization reactions may comprise, for example, fluorescent in-situ hybridization (FISH), DNA paint, multi-barcode identification (e.g., MER-FISH).

The sequencing reactions or hybridization reactions may comprise one or more capture probes or libraries of capture probes. At least one of the one or more capture probe libraries may comprise one or more capture probes to 1, 2, 3, 4, 5, 6, 7, 8, 9, 10, 11, 12, 13, 14, 15 or more genomic regions. The libraries of capture probes may be at least partially complementary. The libraries of capture probes may be fully complementary. The libraries of capture probes may be at least about 5%, 10%, 15%, 20%, %, 25%, 30%, 35%, 40%, 45%, 50%, 55%, 60%, 70%, 80%, 90%, 95%, 97%, or more complementary.

The methods and systems disclosed herein may further comprise conducting one or more sequencing reactions or hybridization reactions on one or more capture probe free nucleic acid molecules. The methods and systems disclosed herein may further comprise conducting one or more sequencing reactions or hybridization reactions on one or more subsets on nucleic acid molecules comprising one or more capture probe free nucleic acid molecules.

The term “label” as used herein is the introduction of a chemical group to the molecule, which generates some form of measurable signal. Such a signal may include, but is not limited to, fluorescence, visible light, mass, radiation, or a nucleic acid sequence.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl group. Any of the above-mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

The term “oxo” refers to C═O.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic group, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g., —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to a divalent heteroalkyl group.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s). In some other embodiments, optional substituents are individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, —CH₂CO₂H, —CH₂CO₂alkyl, —CH₂C(═O)NH₂, —CH₂C(═O)NH(alkyl), —CH₂C(═O)N(alkyl)₂, —CH₂S(═O)₂NH₂, —CH₂S(═O)₂NH(alkyl), —CH₂S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups. In some embodiments, an optional substituent on an aliphatic carbon atom (acyclic or cyclic) includes oxo (═O).

As described herein, the term “handle” refers to a molecule that can couple to the C-terminal carboxylic acid of a protein or peptide. A handle may comprise a backbone (e.g., alkylene, polyethylene glycol, and amide groups), a nucleophile (e.g., amine or thiol), an electrophile (e.g., Michael acceptor), a detection unit (e.g., fluorophore, nucleic acid oligomer, or charged group), a functionalization unit (e.g., biotin, azide, alkyne, thiol, alkene, carboxylic acid, or amine), or any combination thereof. A handle may comprise at least one linker.

A “linker”, as described herein, couples at least two molecules. In some embodiments, a linker couples at least two molecules directly or indirectly. A linker may be a bifunctional molecule for labeling amino acid side chains. One end of the molecule may comprise an amino acid specific functional group (e.g., iodoacetamide for labeling thiol residues on cysteines) and the other end may be a different functional group amenable for labeling. If no reporter is required to be attached, then the functional group may be an inert group (e.g., alkane). The reporter end of the tag molecule may be a fluorophore. A tag may comprise at least one charged molecule that can produce a distinct signal (e.g., fluorescent or electric).

The term “reporter” or “tag” refers to a molecule that produces an identifiable signal. Examples of a reporter include fluorophores (e.g., a cluster of fluorophores), DNA molecules that can be hybridized, or molecules that produces a distinct electrical signal state.

The term “reactive agent”, as used herein, generally refers to a chemical or biological agent that reacts with a peptide or protein. The “reactive agent” may react selectively with the C-terminal amino acid of a peptide or protein.

The term, “internal amino acid residue”, as used herein, generally refers to an amino acid residue between a C-terminal amino acid residue or an N-terminal amino acid residue of a peptide or protein.

The term, “nucleophile”, as used herein, generally refers to a chemical species (e.g., a first atom) that donates an electron pair to form a chemical bond with another chemical species (e.g., a second atom). Examples of atoms that can act as nucleophiles are halogens (e.g., fluoride, chloride, bromine, iodine), oxygen, sulfur, nitrogen, and carbon. Examples of nucleophiles include, but are not limited to, electron rich chemical species, negatively charged chemical species, amines, alcohols, thiols, sulfides, alkynes, alkenes, carboxylic acids, nitriles, water, azides, nitrites, hydroxylamines, hydrazines, and carbazides. The term, “electrophile”, as used herein, generally refers to a chemical species (e.g., a first atom) that accepts an electron pair to form a chemical bond with another chemical species (e.g., a second atom). Examples of atoms that can act as electrophiles are hydrogen, halogens, sulfur, and carbon. Examples of electrophiles include, but are not limited to, electron poor chemical species, positively charged chemical species, alkenes, dienes, acylates, acrylamides, cyanates, carboxylic acids, amides, esters, sulfones, aldehydes, and conjugated systems (e.g., a Michael acceptor or a conjugated aromatic system). For example, a nucleophile can react with an electrophile to form a chemical bond between the nucleophile and the electrophile.

The term, “functionalization moiety”, as used herein, generally refers to a chemical species that is attached to a parent molecule, and which can be chemically modified to provide a way to manipulate the parent molecule.

The term, “enrichment moiety”, as used herein, generally refers to a chemical species that is attached to a parent molecule, and which can be chemically modified to provide a way to increase the relative amount of the parent molecule in a sample.

Compounds

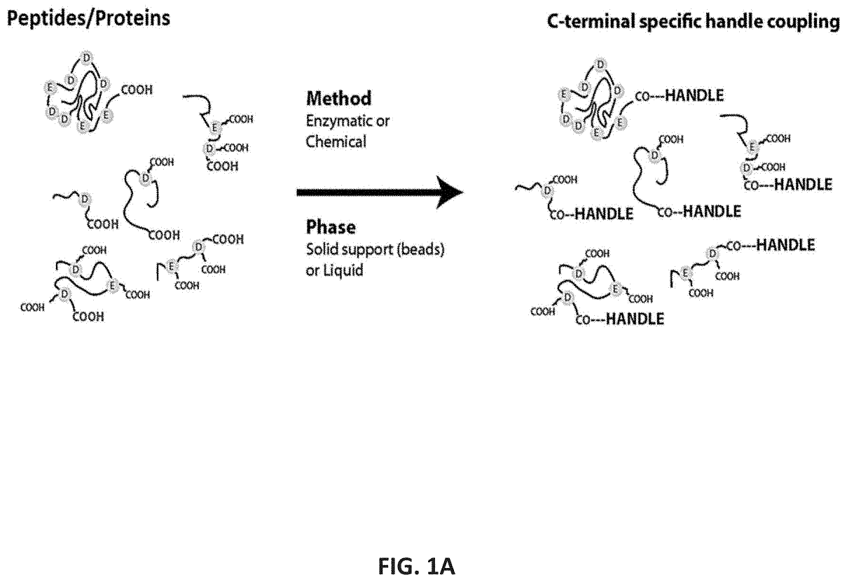

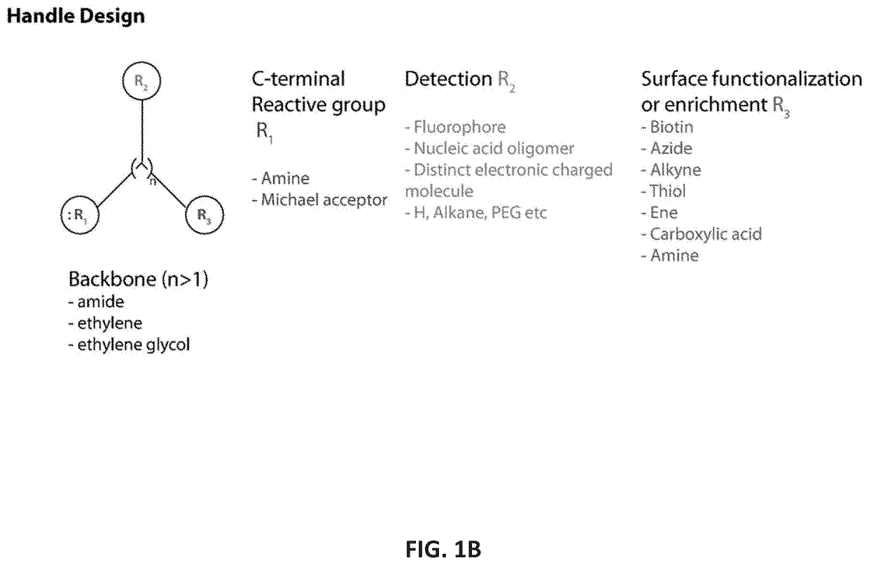

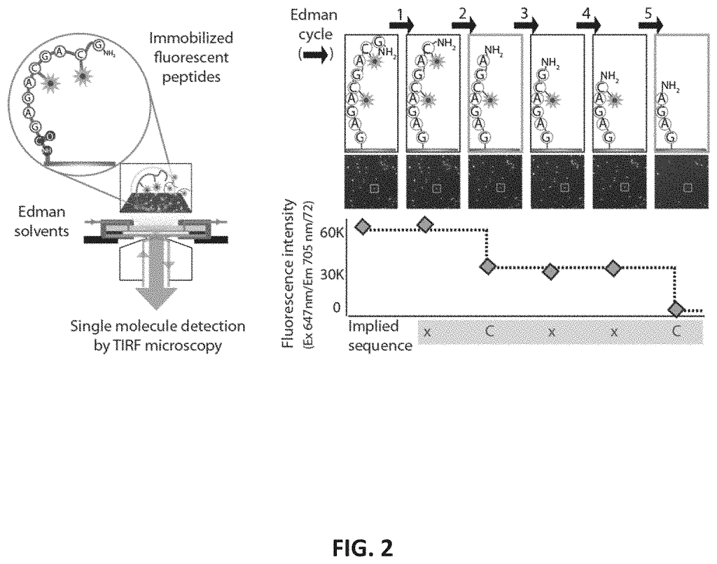

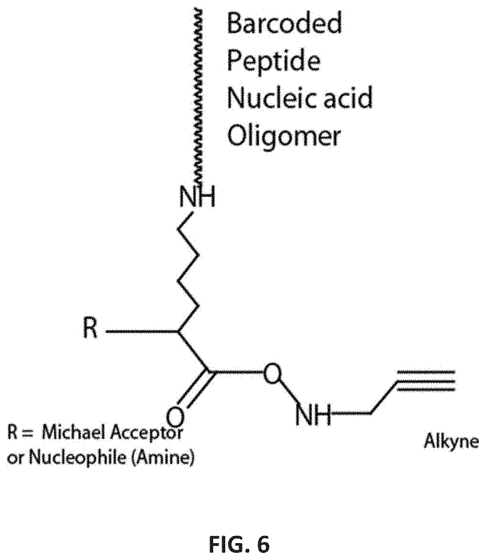

The present disclosure provides C-terminal coupling reagents for labeling a C-terminal amino acid. A C-terminal coupling reagent may comprise (i) a moiety which selectively couples (e.g., forms a covalent bond) to a peptide C-terminal carboxylate, such as a nucleophile (e.g., oxazolone- or enzymatic-type nucleophile (e.g., amine)) or a Michael acceptor (e.g., photoredox-type Michael acceptor) and (ii) at least one functional handle for surface immobilization and/or enrichment of C-terminal peptides (e.g., an alkyne, an azide, biotin, or a nucleic acid (e.g., RNA, DNA, and PNA)) (FIG. 1B). The C-terminal coupling reagents may comprise a peptide or a nucleic acid. The peptide or nucleic acid may comprise at least one internal amino acid chain comprising at least one functional group (e.g., a nucleic acid oligomer, fluorophore, alkyne, azide, and biotin). The Peptide may comprise at least 1, 2, 5, 10, 20, 30, 40, 50, 60, 70, 80, 90, 100, or more amino acids. The Peptide may comprise at least 1, 2, 5, 10, 20, 30, 40, 50, 60, 70, 80, 90, 100, or more functional groups. A functional group can be an inert group such as, such as an alkane, or a reactive functional group, such as a thiol. The peptide or nucleic acid may be a peptide or nucleic acid barcode. A plurality of C-terminal coupling reagents may comprise a plurality of barcodes, for example to enable relative quantification of proteins between samples, control for batch effects. Examples of designs of C-terminal coupling reagents described herein are shown in FIG. 1B and FIG. 6.

Various aspects of the present disclosure provide compositions comprising a peptide coupled to a C-terminal coupling reagent and immobilized to a solid support. The peptide may be coupled to the solid support by the C-terminal coupling reagent (e.g., the C-terminal coupling reagent may be coupled to the peptide and to the solid support), by its N-terminus, or by an internal amino acid residue (e.g., a cysteine thiol of the peptide may be coupled to a maleimide linker coupled to the solid support).

The C-terminal coupling reagent may contain one, two, three or handles. A handle may impart a property (e.g., fluorescence or a charge) to the C-terminal coupling reagent. A handle may be configured for detection (e.g., may comprise a detectable moiety such as a fluorophore), surface immobilization (e.g., may comprise an alkyne configured to couple with a substrate-immobilized azide), enrichment (e.g., may comprise a protein purification tag such as a His-tag or a FLAG-tag), nanopore sequencing (e.g., may comprise a moiety comprising multiple positively charged residues to enhance electrical gradient-mediated migration), or chemical coupling (e.g., copper mediated metathesis to a species of interest, such as a fluorophore), or any combination thereof. A handle may be linked to the C-terminal coupling reagent through one or more linkers (e.g., an oligo ethylene glycol linker).

The C-terminal coupling reagent can be configured for surface immobilization. For example, the C-terminal coupling reagent may comprise a handle comprising an alkyne group configured to couple to an azide group on the functionalized surface, thereby enabling coupling to said surface through a selective reaction (e.g., immobilization may only occur between C-terminal coupling reagent coupled peptides and surface bound azide groups). A C-terminal coupling reagent may comprise a handle configured for click chemistry, a Diels Alder reaction, thiol-ene chemistry, amide coupling or any combination thereof.

Certain aspects disclosed herein provide a compound for labeling a peptide or protein comprising a C-terminus, which comprises a first carboxylic acid moiety, and an internal amino acid residue, which comprises a second carboxylic acid moiety, wherein said compound is configured to preferentially couple to said first carboxylic acid moiety over said second carboxylic acid moiety, wherein the compound has the structure of Formula (I):

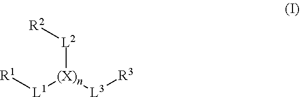

wherein:
L¹, L², and L³ are independently substituted linkers, unsubstituted linkers, or bonds;
R¹ comprises a C-terminal coupling reagent;
R² comprises a handle comprising a detectable moiety;
R³ comprises a handle comprising an enrichment moiety;
each instance of X is independently selected from C—H, an amino acid, or a nucleotide, and n is 1-12.
In some cases, R¹ comprises a C-terminal coupling reagent configured to selectively couple to a peptide C-terminal carboxylate over a carboxylate-containing amino acid side chain (e.g., a glutamate or aspartate side chain).
In some cases, R² and L² are absent (e.g., replaced by hydrogen or an alkane). In some cases, R³ and L³ are absent. In some cases, R², R³, L², and L³ are absent.
In some cases, the compound comprises multiple instances of -L²-R², wherein different instances of -L²-R² may be different or identical.

The compound may have the structure of Formula (Ia):

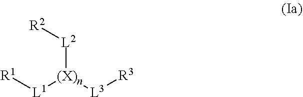

wherein:
L¹, L², and L³ are independently a bond, substituted or unsubstituted alkylene, substituted or unsubstituted alkenyl, substituted or unsubstituted alkynyl, substituted or unsubstituted heteroalkyl, —(R⁴)O(R⁴)—, oxo, —(R⁵)N(R⁶)(═O)(R⁵)—;
R¹ is a C-terminal coupling reagent;
R² is a detection moiety, a reactive agent, or any combination thereof;
R³ is a surface functionalization or surface enrichment moiety;
each instance of X is independently selected from C—H, an amino acid, or a nucleotide,
R⁴ is bond, H, substituted or unsubstituted alkylene, substituted or unsubstituted alkenyl, substituted or unsubstituted alkynyl, or substituted or unsubstituted heteroalkyl;
R⁵ is bond, H, substituted or unsubstituted alkylene, substituted or unsubstituted alkenyl, substituted or unsubstituted alkynyl, substituted or unsubstituted heteroalkyl;
R⁶ is H or substituted or unsubstituted alkyl; and n is 1-12.

In some cases, R¹ comprises a nucleophile. In some cases, the nucleophile comprises an amine, an alcohol, a sulfide, a negatively charged species, or any combination thereof. In some cases, the amine is a primary amine. In some cases, the amine is a secondary amine. In some cases, the amine is a tertiary amine. In some cases, the alcohol is a primary alcohol. In some cases, the alcohol is a secondary alcohol. In some cases, the alcohol is a tertiary alcohol. In some cases, R¹ comprises an electrophile. In some cases, the electrophile is selected from the group consisting of a Michael acceptor, an alkene, a diene, an acrylamide, an N-(prop-2-yn-1-yl)methylacrylamide, an isocyanate, an isothiocyanate, an oxirane, an α, β-unsaturated carbonyl, a vinyl sulfone, a norbornanone, or any combination thereof. In some cases, R¹ comprises a Michael acceptor. The Michael acceptor may comprise an α,β-unsaturated ketone, an α,β-unsaturated carboxylate, an α,β-unsaturated ester, an α,β-unsaturated amide, an α,β-unsaturated nitrile, a nitroalkene (e.g., 2-nitrobicyclo[2.2.1]hept-2-ene), an α,β-unsaturated sulfone, or any combination thereof. The Michael acceptor may be a sterically constrained Michael acceptor (e.g., the Michael α,β-unsaturated positions may be disposed within a bicyclic group, such as bicycloheptane).[Please edit it accordingly—strained, monocarbonyl-containing compound could be a general compound name that acts on the C-terminus using the photoredox chemistry]

Various aspects of the present disclosure provide C-terminal coupling reagents comprising a Michael acceptor comprising a bridged polycyclic alkyl or heteroalkyl structure. Such Michael acceptors may impart enhanced selectivity toward C-terminal carboxyl groups (e.g., over aspartate and glutamate side chain carboxyl groups) due to their steric bulk and, in some cases, lower reactivities. A bridged polycyclic structure may comprise an optionally substituted bridged bicyclic C₅-C₁₄ structure, such as bicyclo[1.1.1]pentane, bicyclo[2.1.1]hexane, bicyclo[2.2.1]heptane, bicyclo[2.2.2]octane, or bicyclo[3.3.1]nonane. A bridged polycyclic structure may comprise an optionally substituted bridged tricyclic structure, such as tricyclo[2.2.1.0^{2,6}]heptane or tricyclo[5.2.1.0^{2,6}]decane. In some cases, the Michael acceptor comprises an electron withdrawing group and a β-unsaturated carbon (e.g., a carbonyl or nitro group) bound directly to the bridged polycyclic structure (e.g., O or

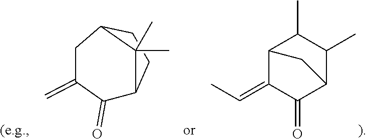

In some cases, the Michael acceptor comprises α,β-unsaturated carbons within the bridged polycyclic structure. For example, the compound may comprise a C-terminally reactive Michael acceptor comprising

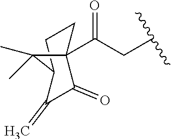 [SMILES: *CC(=O)C12CCC(C(=C)C1=O)C2(C)C]

or a derivative thereof.

In some cases, the Michael acceptor comprises a structure of formula (II), or a salt, solvate, tautomer, or N-oxide thereof:

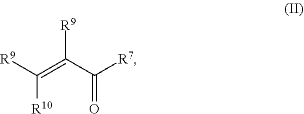

wherein R⁷ and R⁸ are taken together to form a bridged bicyclic or tricyclic C₅-C₁₄ alkyl or heteroalkyl structure optionally substituted with one or more instances of R¹¹;
R⁹, R¹⁰, and each instance of R¹¹ are independently selected from the group consisting of hydrogen, halogen, hydroxyl, optionally substituted aryl, optionally substituted heteroaryl, optionally substituted cycloalkyl, optionally substituted heterocycloalkyl, optionally substituted amine, —C(═O)—R¹², optionally substituted alkyl, optionally substituted alkenyl, optionally substituted alkynyl, optionally substituted alkoxy, optionally substituted haloalkyl, optionally substituted heteroalkylene, and optionally substituted haloalkoxy, or R⁹ and R¹⁰ are taken together to form a cycloalkyl, a heterocycloalkyl, an aryl, or a heteroaryl; and
each instance of R¹² is independently selected from the group consisting of hydrogen, halogen, hydroxyl, optionally substituted alkyl, optionally substituted hydroxyalkyl, optionally substituted heteroalkylene, optionally substituted alkoxy, optionally substituted haloalkyl, and optionally substituted haloalkoxy.

In some cases, R⁷ and R⁸ are taken together to form a bridged bicyclic C₅-C₁₄ alkyl or heteroalkyl structure optionally substituted with one or more instances of R¹¹. In some cases, R⁷ and R⁸ are taken together to form a bridged bicyclic C₆-C₁₀ alkyl or heteroalkyl structure optionally substituted with one or more instances of R¹¹. In some cases, R⁷ and R⁸ are taken together to form a bridged bicyclic C₇-C₉ alkyl or heteroalkyl structure optionally substituted with one or more instances of R¹¹. In some cases, R⁷ and R⁸ are taken together to form a bridged bicyclic C₇-C₉ alkyl or heteroalkyl structure substituted with at least one instance of R¹¹. In some cases, R⁷ and R⁸ are taken together to form a bridged bicyclic C₈-C₁₀ alkyl or heteroalkyl structure substituted with at least one instance of R¹¹.

In some cases, R⁹, R¹⁰, and each instance of R¹¹ are independently selected from the group consisting of hydrogen, halogen, hydroxyl, —C(═O)—R¹², optionally substituted alkyl, optionally substituted alkenyl, optionally substituted alkynyl, optionally substituted alkoxy, and optionally substituted haloalkyl. In some cases, at least one of R⁹ and R¹⁰ is hydrogen. In some cases, at least one of R⁹ and R¹⁰ is not hydrogen. In some cases, R⁹ and R¹⁰ are hydrogen. In some cases, each instance of R¹¹ is not hydrogen. In some cases, each instance of R¹¹ is selected from the group consisting of C₁-C₄ alkyl. In some cases, each instance of R¹¹ is methyl.

In some cases, optionally substituted denotes hydroxyl, halogen, —NH₂, alkyl, alkenyl, or alkynyl substitution. In some cases, optionally substituted denotes hydroxyl, —NH₂, or alkyl substitution.

In some cases, the Michael acceptor comprises a norbornenone moiety or a derivative thereof. In some cases, the norbornenone comprises a methylene norbornanone or a derivative thereof. In some cases, the Michael acceptor comprises 3-methylene-2-norbornanone or a derivative thereof.

### Methods for C-Terminal Modification

Proteins, peptides, or combinations thereof may comprise a C-terminal amino acid residue. Proteins, peptides, or combinations thereof can derive from, for example, cell lysate, biological fluid (e.g., blood, plasma, urine, saliva), or combinations thereof. The proteins, peptides, or combinations thereof can be recombinant, synthetic, or a combination thereof. Proteins, peptides, or combinations thereof can be enriched using, for example, antibody pull down methods (e.g., immunoprecipitation), affinity pull-down methods, Glutathione-S-transferase (GST) pull-down methods, tandem affinity purification (TAP) methods, or any combination thereof. The proteins, peptides, or combinations thereof can be extracted by protein isolation methods (e.g., chromatography and electrophoresis). Peptides, proteins, or combinations thereof may be generated from cells, biological fluids, or combinations thereof, and can be separated using chromatography (e.g., size-exclusion, ion-exchange, and affinity-based) or other gel-based extraction methods (e.g., agarose).

Proteins, peptides, or combinations thereof may be digested into peptide fragments of the proteins, peptides, or combinations thereof. Digestion may be accomplished by, for example, enzymes or small molecules (e.g., cyanogen bromide, NTCB (2-nitro-5-thiobenzoic acid, and isothiocyanates). The enzymes may be proteolytic enzymes. The enzymes may be endo-proteolytic enzymes (e.g., trypsin and Glu-C). A peptide fragment derived from the proteins, peptides, or combinations thereof may contain a C-terminal amino acid comprising a terminal carboxylate. The digestion methods disclosed herein may generate peptide fragments of various lengths. A method may generate peptide fragments with an average length of at least 10 amino acids, at least 12 amino acids, at least 15 amino acids, at least 20 amino acids, at least 25 amino acids, at least 30 amino acids, at least 40 amino acids, at least 50 amino acids, at least 60 amino acids, at least 70 amino acids, or at least 80 amino acids. For example, a digestion method may comprise a single mutant protease that generates peptide fragments with average lengths of 55-70 amino acids. A method may generate peptide fragments with an average length of at most 80, at most 70, at most 60, at most 50, at most 40, at most 30, at most 25, at most 20, at most 15, at most 10, at most 8, or at most 5 amino acids. For example, a digestion method may comprise trypsinization, and may thereby generate peptide fragments with an average length of between 7 and 15 amino acids.

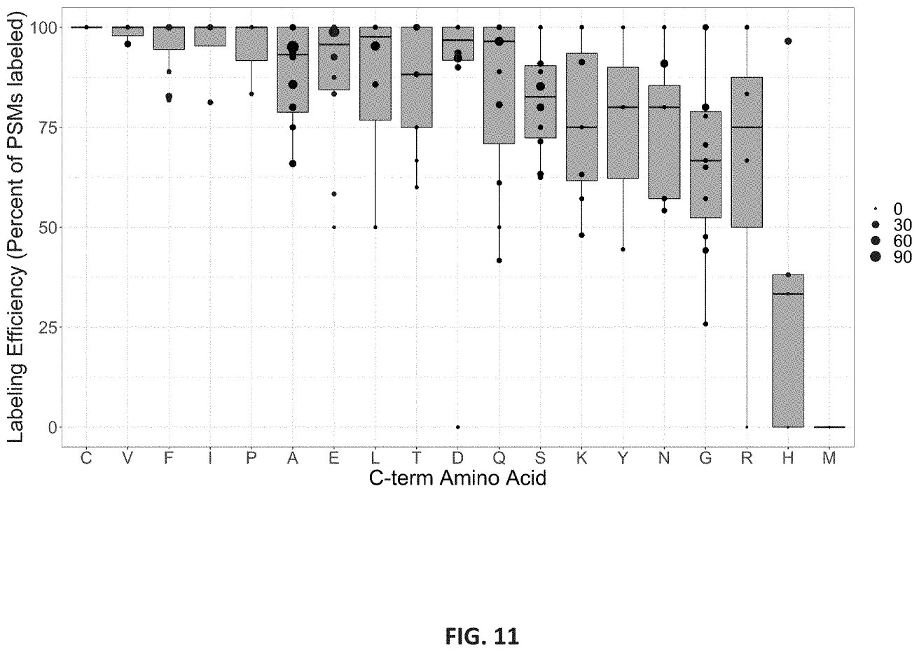

A method may generate peptide fragments comprising identical C-terminal amino acids. A challenge in selective C-terminal labeling stems from variable amino acid-type affinities exhibited by some C-terminal coupling reagents. A C-terminal coupling reagent may comprise a range of affinities for different types of C-terminal amino acids. For example, as is shown in FIG. 11, a norbornenone C-terminal coupling reagent may comprise a high affinity for cysteine and valine C-terminal amino acid carboxyl groups, and a relatively low affinity for histidine C-terminal amino acid carboxyl groups. Accordingly, a method may comprise GluC digestion, and thereby be configured to generate peptide fragments with glutamic acid and aspartic acid C-termini. A method may comprise enterokinase or thrombin digestion, and thereby be configured to generate peptide fragments with lysine C-termini. A method may comprise factor Xa digestion, and thereby be configured to generate peptide fragments with arginine C-termini. A method may comprise TEV protease digestion, and thereby be configured to generate peptides with glutamine C-termini.

The proteins, peptides, or combinations thereof may comprise reactive amino acid residues (e.g., internal amino acid side chain residues, N-terminal amino acid amine or side chain residue). A reactive amino acid residue of a protein, peptide, or combinations thereof may be protected (e.g., reversibly coupled to a protecting reagent to diminish the reactivity of the reactive amino acid residue). A reactive amino acid residue may be protected prior to the labeling of a C-terminal amino acid. The reactive amino acid residue may be reversibly or irreversibly reacted. Protecting reactive amino acid residues may prevent or eliminate the formation of side-products that can form during a C-terminal labeling reaction. Reactive amino acids may be modified prior to or after isolation of a protein, peptide, or combination thereof. Modifications prior to isolation of a protein, peptide, or a combination thereof may be a post-translational modification. Post-translational modifications may include, for example, phosphorylation, ubiquitinoylation, methylation, acetylation, acylation, carboxylation, nitrosylation, citrullination, or any combination thereof. Reactive amino acid residues may include, for example, cysteine, N-terminus, lysine, tyrosine, serine, threonine, arginine, histidine, aspartic acid, glutamic acid, glutamine, proline, and tryptophan.

Examples of blocking nucleophilic side chains before or after C-terminal labeling include:
a) Cysteine: Thiol groups on the Cysteine residues may be reversibly or irreversibly labeled with a cysteine reactive linker such as an iodoacetamide- or maleimide-containing compound.
b) N-terminal amino acid: The amino group at the N-terminus of the proteins, peptides, or combinations thereof may be selectively blocked via an electrophile (e.g., pyridine carboxaldehyde (PCA)). The N-terminus may be blocked in either the liquid or solid phase (e.g., the electrophile is tethered to a solid support). The N-terminal amino group can be blocked to afford a reversible protecting group.
c) Lysine: The amine side chain can be labeled with a succinimidyl ester, a lysine-selective methyltransferase, a vinyl sulfone, a carbamate, a thiocarbamate, a carbonate, a thiocarbonate, sulfonyl chloride, Tetrafluorophenyl (TFP) Esters, carbonyl azides, aldehydes or any combination thereof.

Other examples of blocking nucleophilic side chains include compositions and methods disclosed in, for example, Basle et al., Protein Chemical Modification on Endogenous Amino Acids, Chemistry and Biology, 17, Mar. 26, 2010. The examples provided herein for blocking nucleophilic side chains are not intended to be limiting. Any nucleophilic amino acid side chain of a peptide or protein can be blocked with reactive agents selective for an amino acid type. It may not be necessary to block amino acid side chains of a peptide or protein to selectively react compositions described herein to the C-terminal amino acid of the peptide or protein.

A protein, peptide, or combination thereof can be released before or after the C-terminus is modified. In some cases, the following can be performed—(1) collect or isolate a plurality of peptides, (2) immobilize the peptides on a solid support (e.g., with cysteine selective capture moieties or PCA-bead capture chemistry (for example by conjugation of an N-terminal amine)), (3) conjugate the peptide C-termini with a C-terminal coupling reagents, (4) label the side-chains of the proteins, peptides, or combinations thereof and (5) release the proteins, peptides, or combinations thereof for downstream analysis.

### Chemical Methods

Various methods of the present disclosure comprise derivatizing a peptide C-terminal prior to coupling a C-terminal coupling reagent. The derivatizing may increase the reactivity of the C-terminal toward the C-terminal coupling reagent. The derivatizing may increase the selectivity of the C-terminal coupling reagent. The derivatizing may be enzymatic. The derivatizing may be non-enzymatic. The derivatizing may comprise a single step (e.g., oxazolone derivatization of a peptide C-terminus) or multiple steps. The derivatizing may comprise C-terminal conversion to an oxazolone intermediate, carbamoylation of the C-terminal, C-terminal conversion to a furandione, C-terminal amidation, C-terminal decarboxylation (e.g., decarboxylative alkylation), or any combination thereof.

A peptide C-terminal may be derivatized to form an oxazolone intermediate, thereby enabling specific C-terminal reactions despite the difficulty in discriminating the C-terminus from Asp/Glu side chains. Current discriminatory methods are limited at least because they (i) have low derivatization efficiency, (ii) do not contain a functionalization moiety or an enrichment moiety (e.g., a bi-functional handle), (iii) do not react to afford substantial yield (e.g., at least about 90%, 95%, 99%, 99.9%, or more C-terminally reacted peptide or protein) to perform proteomics (e.g., sequencing), (iv) require use of organic reagents and high temperatures that are not amenable for peptides, proteins, or combinations thereof, and/or (v) do not provide substantial specificity (at least about 10:1, 100:1, 1,000:1, or more specificity for the C-terminal amino acid residues compared to internal amino acid residues) over Asp/Glu to perform proteomics (e.g., sequencing). A number of methods and compositions disclosed herein provide an adapted form of C-terminus selective oxazolone ring formation to allow for the attachment of a bi-functional handle to the C-terminus without reacting to the internal acidic groups on aspartate or glutamate residue.

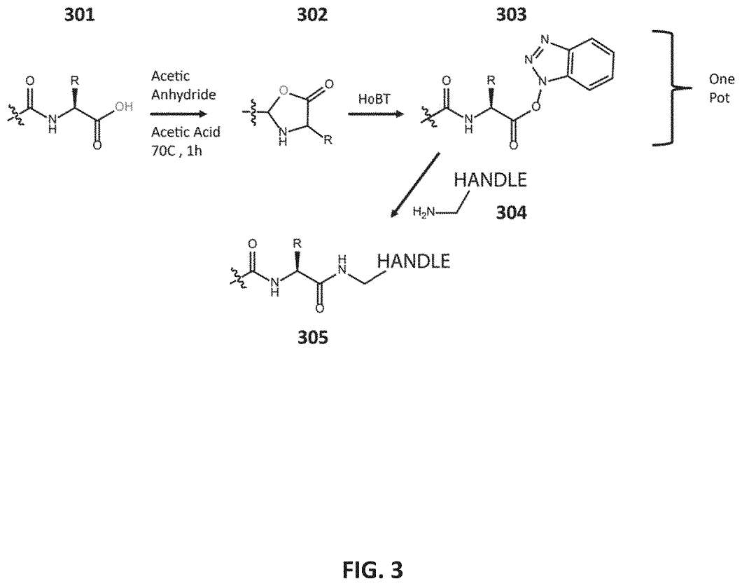

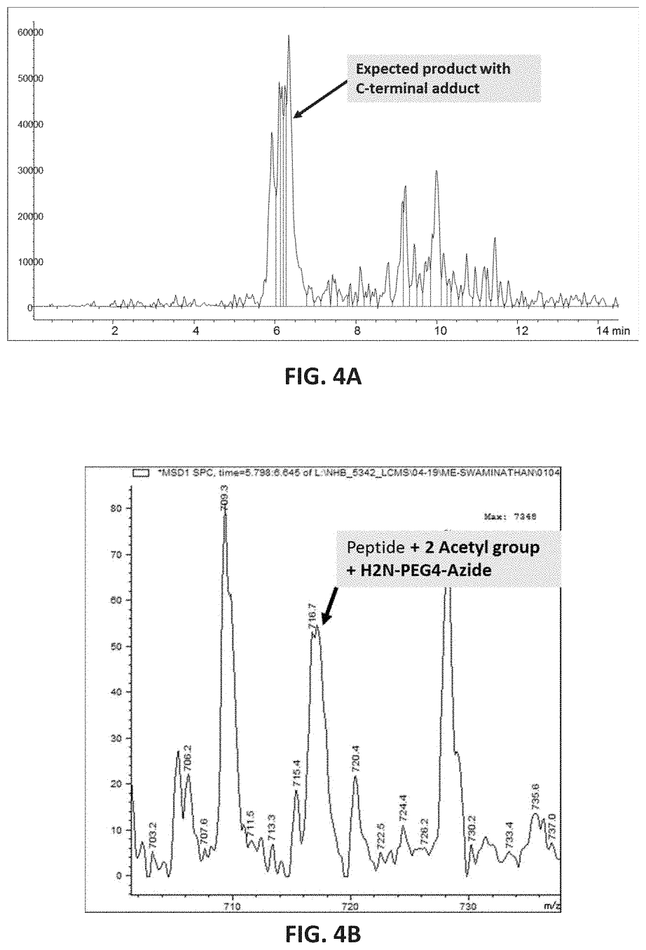

The oxazolone ring may be directly reacted with a C-terminal coupling reagent, or may be activated (e.g., by coupling to hydroxybenzotriazole (HoBT)) prior to reaction with a C-terminal coupling reagent. Activating an oxazolone intermediate may increase the yield and specificity of a coupling step comprising a C-terminal coupling reagent and a peptide C-terminus. For example, activating an oxazolone intermediate may increase its electrophilicity, thereby enabling the use of lower nucleophilicity (and therefore lower cross-reactivity and higher specificity) C-terminal coupling reagents. An example of such a mechanism is illustrated in FIG. 3.

A method of the present disclosure may comprise directly reacting a C-terminal coupling reagent with a peptide C-terminal. An example of a chemical method that can be configured to discriminate the carboxylic group of a peptide C-terminus is photoredox chemistry. Accordingly, the present disclosure provides photoredox methods and reagents (e.g., photoredox catalysts) optimized for selective C-terminal labeling of peptides and proteins (e.g., insulin). A photoredox catalyst or method may discriminate between internal versus C-terminal carboxylates based on their differences in the reduction potential (e.g., the C-terminal may be more readily reducible than an internal carboxylate residue). For example, a flavin photocatalyst may comprise an at least 3-fold specificity, at least 5-fold specificity, at least 8-fold specificity, at least 10-fold specificity, at least 12-fold specificity, at least 15-fold specificity, at least 20-fold specificity, at least 25-fold specificity, at least 50-fold specificity, at least 100-fold specificity, or at least 200-fold specificity for a C-terminal carboxylate over a carboxyl side chain.

Photocatalyst activation may be optimized for C-terminal selectivity. In some cases, photocatalyst activation may be achieved with relatively low power light, thereby minimizing non-selective, promiscuous photocatalyst behavior. For example, photocatalyst activation may be achieved with less than 2 watt (W) light, less than 1.5 W light, less than 1 W light less than 750 mW light, less than 500 mW light, less than 400 mW light, less than 300 mW light, less than 200 mW light, less than 150 mW light, less than 120 mW light, less than 100 mW light, less than 80 mW light, less than 60 mW light, or less than 50 mW light. Similarly, utilizing narrow bandwidth (e.g., the full width at half maximum intensity) light for photocatalyst activation may enhance C-terminal carboxylate selectivity. Accordingly, photocatalyst activation may be achieved with less than 60 nm bandwidth light (e.g., 390-490 nm light from a photoexcitation source such as a lamp), less than 50 nm bandwidth light, less than 40 nm bandwidth light, less than 30 nm bandwidth light, less than 25 nm bandwidth light, less than 20 nm bandwidth light, less than 15 nm bandwidth light, less than 12 nm bandwidth light, less than 10 nm bandwidth light, less than 8 nm bandwidth light, less than 6 nm bandwidth light, less than 5 nm bandwidth light, less than 3 nm bandwidth light, or less than 2 nm bandwidth light. A light source may comprise a filter (e.g., a narrow band-pass optical filter) to control the bandwidth of light reaching a sample. A light source may provide light with a central wavelength of 350 nm to 550 nm, 400 nm to 700 nm, 350 nm to 400 nm, 400 nm to 450 nm, 450 nm to 500 nm, 500 nm to 550 nm, or 550 nm to 600 nm. For example, a photocatalysis method may utilize a 450 nm (blue) LED light source with 220 mW of power and a bandwidth of 25 nm. Illumination may be performed for at least 0.25 hours, at least 0.5 hours, at least 0.75 hours, at least 1 hour, at least 1.5 hours, at least 2 hours, at least 2.5 hours, at least 3 hours, at least 3.5 hours, at least 4 hours, at least 5 hours, at least 6 hours, at least 7 hours, at least 8 hours, at least 9 hours, at least 10 hours, at least 11 hours, or at least 12 hours.

A Michael acceptor for photoredox chemistry may be, for example, a substituted or unsubstantial norbornanone, a malonate, or a maleimide. The Michael acceptor may be, for example, a norbornenone variant, 3-methylene-2-norbornanone, diethyl ethylidenemalonate, or maleimide. Other Michael acceptors may include, for example, a substituted alkene, a diene, an acrylamide, an N-(prop-2-yn-1-yl)methylacrylamide, an isocyanate, an isothiocyanate, an oxirane, an α, β-unsaturated carbonyl, a norbornanone, a vinyl sulfone, or any combination thereof.

### Enzymatic Methods

C-terminal labeling may comprise enzymatic ligation. The principle of the enzymatic ligation strategy is to repurpose the cleavage property of endo- and exopeptidases to perform peptide ligation (e.g., by coupling an appropriate nucleophile under an altered enzyme conformation). Enzymes can have varying degrees of specificity for different amino acid types. An enzyme (e.g., carboxypeptidase Y) can have broad specificity for C-terminal amino acids or may have strict requirements for C-terminal amino acid type (e.g., thermolysin). Other classes of modifying enzymes (e.g., amidases) may be used for C-terminal labeling.

Described herein are methods comprising enzymatic labeling of the carboxyl termini of a donor (e.g., peptides, proteins, or a combination thereof) with an acceptor (e.g., a fixed molecular adaptor such as a C-terminal coupling reagent). The activity of an enzyme may be dependent on or independent of the type of C-terminal amino acid on a target peptide. For example, carboxypeptidase enzyme can exhibit C-terminal amino acid-type independent activity. Conversely, peptiligase enzymes (e.g., the Omniligase variant Thymosin-alpha-1) can comprise C-terminal amino acid-type dependent activity (e.g., no reactivity toward peptides containing proline C-termini, high activity toward peptides containing zwitterionic lysine and arginine C-termini. The N-terminal ligase activity of a peptiligase enzyme may be repurposed for a C-terminal labeling reaction of peptides, proteins, or combinations thereof.

Carboxypeptidase Y is a yeast serine protease commonly used for removal of C-terminal amino acids, and it can have transpeptidase activity. The carboxypeptidase may mediate ligation of a nucleophilic handle to the C-terminal of proteins, peptides, or a combination thereof. The ligation may involve selective and positive enrichment for only C-terminal peptides of a proteins, peptides, or combinations thereof. The methods and compositions described herein can be adapted to attach the nucleophilic handle to the C-terminal of peptides, proteins, or combinations thereof.

Omniligase is an engineered subtiligase that can perform a transpeptidation reaction and is sold by EnzyPep B.V (Geleen, Netherlands). The intramolecular ligation reaction may involve the reaction of an acyl modified amino acid esters (e.g., substituted Cam-ester), making up the C-terminal end of the donor peptide or protein with a free N-terminal amine of the acceptor peptide or protein. There may be biases in the amino acid choice for an efficient ligation reaction. This bias may reduce the number of peptides or proteins ligated but can carry the information of the permissible amino acid sequences comprising the donor or acceptor peptide or protein molecule. The Omniligase reaction is described herein and may be used for ligating a constant “acceptor” handle to the N-termini of individual peptides, proteins, or a combination thereof.

The ligation activity of the Omniligase reactivity may be repurposed to ligate the C-termini of each peptide, protein, or combination thereof in a heterogeneous pool with a constant nucleophilic handle (acceptor). This can be accomplished by activating the acidic ends of the peptide or protein to an ester form (e.g., alkyl ester or Cam-ester). The acidic ends may be activated to an ester form with methanolic HCl. After a linker is attached, the Asp/Glu side chains may be capped as esters. The esters of the peptides or proteins can be hydrolyzed under high pH (pH 12) to reveal the standard acidic side chains. The transpeptidation reaction can be carried out in solid phase immobilized peptides or proteins or in the liquid phase. The transpeptidation reaction can be carried out in the liquid phase.

Peptide or protein immobilization can be achieved using the side chain of the C-terminal amino acid residue. For example, in the case of chemical digestion of the protein lysate with NTCB (2-nitro-5-thiobenzoic acid), the peptides, proteins, and combinations thereof may have cysteine as the C-terminal amino acid residue. The thiol-containing sidechain can be functionalized with a handle that comprises an iodoacetamide group and an appropriate functional group for surface immobilization. As another example, in a case of peptides with lysine at the C-terminal, following trypsin digests, can be immobilized to the surface via the F-amine reacting to the handle. In these methods, the acidic residues on glutamate, aspartate, and the C-terminal amino acid are available for reaction. A method of the present disclosure may thus comprise immobilizing a peptide to a surface by an internal amino acid residue, the N-terminal amino acid terminal amine or side chain, or the C-terminal amino acid side chain, and coupling the C-terminal amino acid to a C-terminal coupling reagent. In some cases, the peptide is immobilized to the surface prior to coupling to the C-terminal coupling reagent. In some cases, the peptide is coupled to the C-terminal coupling reagent prior to the immobilizing to the surface.

### Methods of Labeling a C-Terminus of a Peptide or Protein

In certain aspects, disclosed herein is a method for processing a peptide or protein comprising a C-terminus, which comprises a first carboxylic acid moiety, and an internal amino acid residue, which comprises a second carboxylic acid moiety, the method comprising coupling said first carboxylic acid moiety with a reactive agent (e.g., a C-terminal coupling reagent) preferentially over said second carboxylic acid moiety. The C-terminal coupling reagent may preferentially couple to the first carboxylic acid moiety over the second carboxylic acid moiety with at least about 10%, at least about 20%, at least about 30%, at least about 40%, at least about 50%, at least about 60%, at least about 70%, at least about 80%, at least about 90%, at least about 95%, at least about 99%, at least about 99.9%, or at least about 99.99% or greater efficiency. The C-terminal coupling reagent may preferentially couple to the first carboxylic acid moiety over the second carboxylic acid moiety with about 10% to about 99.99%, about 50% to 99.99%, about 90% to about 99.99%, or 95% to 99.99% efficiency. The reactive agent may not react with the second carboxylic acid moiety. The reactive agent may only react with the first carboxylic acid moiety. In some cases, the peptide or protein does not comprise the second carboxylic acid moiety. The peptide or protein may comprise amino acid residues that do not comprise a carboxylic acid side chain.

In certain aspects, disclosed herein is a method for processing a peptide or a protein comprising a C-terminus, which comprises a first carboxylic acid moiety, and an internal amino acid residue, which comprises a second carboxylic acid moiety, the method comprising coupling a reactive agent (e.g., a C-terminal coupling reagent) to said first carboxylic acid moiety in the absence of coupling said reactive agent to said second carboxylic acid moiety. The peptide or protein may comprise at least about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1,000, or more internal amino acid residues. The peptide or protein may comprise at most about 1,000, 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or less internal amino acid residues. The peptide or protein may comprise from about 2 to about 1,000, about 10 to about 100, or about 10 to about 50 internal amino acid residues. At least one or more of the at least two internal amino acid residues may comprise the second carboxylic acid moiety. For example, if a peptide or protein comprises 100 internal amino acid residues, 1, 2, 3, 4, 5, 6, 7, 8, 9, 10, 15, 20, 30, 40, 50, or more of the 100 internal amino acid residues may comprise the second carboxylic acid moiety.

In certain aspects, described herein is a method for processing a peptide or protein comprising a C-terminus, which comprises a first carboxylic acid moiety, and an internal amino acid residue, which comprises a second carboxylic acid moiety, the method comprising coupling said first carboxylic acid moiety of said immobilized peptide or protein with a C-terminal coupling reagent preferentially over said second carboxylic acid moiety of said immobilized peptide or protein. In some cases, the peptide or protein is immobilized to a surface such as a slide (e.g., a microscope slide), a bead, or a surface of a well plate well.

In certain aspects, described herein is a method for processing a peptide or protein comprising a C-terminus, which comprises a first carboxylic acid moiety, and an internal amino acid residue, which comprises a second carboxylic acid moiety, the method comprising coupling said first carboxylic acid moiety of said peptide or protein with a C-terminal coupling reagent preferentially over said second carboxylic acid moiety of said peptide or protein, wherein said reactive reagent comprises a functionalization moiety, an enrichment moiety, or a combination thereof.

A C-terminal coupling reagent may comprise a handle. The handle may comprise an optical label, such as, for example, a fluorescent dye, a quantum dot, a luminescent dye, or a FRET acceptor or donor. The handle may comprise a nucleic acid molecule, such as, for example, a DNA barcode or a DNA points accumulation for imaging in a nanoscale topography (DNA-PAINT) assay. The handle may comprise an ionizable molecule, such as, for example, a tandem mass tag (TMT) or an isobaric tag. The handle may comprise an electrochemically detectable label (e.g., a moiety comprising a characteristic reduction or oxidation potential, such as ferrocene). The handle may comprise a polyethylene spacer. The handle may comprise a polyarginine peptide. The handle may comprise an optical label (e.g. fluorophore), a nucleic acid molecule (e.g., DNA, RNA, PNA), an ionizable molecule (e.g., a bromine, an amine, a phosphate), a polyethylene spacer, a polyarginine peptide, or any combination thereof.

A C-terminal coupling reagent may comprise a carboxylate capture moiety, such as a nucleophile (e.g., a primary amine). A C-terminal coupling reagent may comprise an electrophile. The reactive agent may comprise a nucleophile and an electrophile. The nucleophile may comprise, for example, an amine, an alcohol, a sulfide, a cyanate, a thiocyanate, a deprotonated atom, or any combination thereof. The electrophile may comprise a Michael acceptor, an alkene, a diene, an acrylamide, an N-(prop-2-yn-1-yl)methylacrylamide, an isocyanate, an isothiocyanate, a conformationally constrained moiety (e.g., an oxirane, an α, β-unsaturated carbonyl, a norbornanone), a vinyl sulfone, or any combination thereof.

A C-terminal coupling reagent may comprise a handle comprising a functionalization moiety, an enrichment moiety, or a combination thereof. The enrichment moiety may enable purification of C-terminal functionalized peptides, for example by affinity chromatography or immunoprecipitation. The functionalization moiety may be configured to couple to a capture reagent, such as a substrate-bound (e.g., bead- or glass slide-bound) capture agent. The functionalization moiety or the enrichment moiety may comprise an alkyne, an azide, a fluorophore, biotin, a nucleic acid molecule (e.g., RNA, DNA, PNA), an amino acid, a peptide (e.g., an epitope such as a FLAG-tag), a solid support bead or resin, or any combination thereof.

A method may comprise treating said peptide or protein with at least one chemical, at least one enzyme, or a combination thereof. The at least one chemical, at least one enzyme, or a combination thereof may selectively activate the C-terminal amino acid residue of the peptide or protein (e.g., for coupling to a C-terminal coupling reagent). The at least one chemical may be a photocatalyst. The photocatalyst may be, for example, a flavin (e.g., riboflavin, lumiflavin). The at least one chemical may react with the C-terminal amino acid of the peptide or protein to form an oxazolone intermediate of said C-terminal amino acid of said peptide or protein. The oxazolone intermediate may be reacted with a C-terminal coupling reagent, or may be activated prior to reaction with the C-terminal coupling reagent. The at least one chemical may be, for example, acetic anhydride, hydroxybenzotriazole (HOBT), hydroxyazabenzotriazole (HOAT), 2-nitro-5-thiobenzoic acid (NTCB), or a combination thereof. The at least one enzyme may be a peptidase, an amindase, a hydrolase, or any combination thereof. The at least one enzyme may be, for example, an endopeptidase, an exopeptidase, a carboxypeptidase, an amidase, a hydrolase, a proteinase, a peptiligase, or any combination thereof. The peptiligase may be Omniligase or a modified derivative thereof. The carboxypeptidase may be, for example, carboxypeptidase A, carboxypeptidase B, carboxypeptidase C, carboxypeptidase Y, or a modified derivative thereof. The carboxypeptidase may be carboxypeptidase Y. The proteinase may be thermolysin or a modified derivative thereof.

The method may comprise cleaving a plurality of peptides or proteins, wherein said plurality of peptides or proteins comprises said peptide or protein. The peptide or protein may not comprise the second carboxylic acid moiety. The plurality of peptides or proteins can comprise at least one peptide or protein with the second carboxylic acid moiety.

A C-terminal coupling reagent may be inert toward (e.g., not substantially couple to) (i) the at least one internal amino acid residue and (ii) an N-terminal amino acid residue of the peptide or protein. A C-terminal coupling reagent may be inert toward the at least one internal amino acid residue of the peptide or protein. The reactive agent may be inert toward an N-terminal amino acid residue of the peptide or protein. A C-terminal coupling reagent may be inert to internal amino acid residues of the peptide or protein. A C-terminal coupling reagent be inert toward internal amino acid residue of the peptide or protein. The at least one internal amino acid residue may be a natural or unnatural amino acid. The said at least one said internal amino acid residue may comprise a functional group selected from the group consisting of an amine, a carboxylic acid, an indole, a primary alcohol, a secondary alcohol, a thiol, a thioether, a phenol, an amide, a guanidine, an imidazole, or any combination thereof. The at least one internal amino acid residue, the N-terminal amino acid residue of said peptide or protein, or a combination thereof may be modified before coupling the reactive agent to the first carboxylic acid moiety. The at least one internal amino acid residue, the N-terminal amino acid residue of said peptide or protein, or a combination thereof may be modified after coupling the reactive agent to the first carboxylic acid moiety. At least 1, 2, 3, 4, 5, 6, 7, 8, 9, 10, 11, 12, 13, 14, 15, 16, 17, 18, 19, 20, or more amino acid types of the peptide or protein may be modified before or after coupling the reactive agent to the first carboxylic acid moiety. At least 1, 2, 3, 4, 5, 6, 7, 8, 9, 10, 11, 12, 13, 14, 15, 16, 17, 18, 19, 20, or more amino acid types and the N-terminal amino acid of the peptide or protein may be modified before or after coupling the reactive agent to the first carboxylic acid moiety. The modified internal amino acid type may be cysteine, lysine, tyrosine, tryptophan, serine, threonine, arginine, or any post-translational modification or combination thereof.

The at least one internal amino acid residue may be coupled to at least one label. A plurality of internal amino acid residues may each be coupled to the at least one label (e.g., 5 labels may be separately coupled to 5 internal amino acid residues). Each internal amino acid of a peptide or protein may be coupled to at least one label. Each internal amino acid of an amino acid type (e.g., lysine, cystine, serine, etc.) of the peptide or protein may be coupled to at least one label. Each internal amino acid of an amino acid type (e.g., lysine, cystine, serine, etc.) of the peptide or protein may be coupled to the same type of labeling reagent. The at least one label may correspond to a different label for different internal amino acid types. For example, every lysine of the peptide or protein may be coupled to a red fluorescent label, while every serine may be coupled to a green fluorescent label. The at least one label may be an optically detectable label. The optical label may be a fluorescent dye or a FRET donor or acceptor. The optical label may be a fluorophore. The at least one label may comprise a lysine-specific label, a cysteine specific label, a carboxylate side chain (e.g., glutamate and aspartate) specific label, a tryptophan specific label, a tyrosine specific label, a histidine specific label, an arginine specific label, a serine specific label, a threonine specific label, or any combination thereof. The at least one label may further comprise a non-natural amino acid (e.g., chlorotyrosine) or post-translationally modified amino acid (e.g., phosphotyrosine) specific label.

The method may further comprise producing a labeled peptide or protein for surface immobilization, sample multiplexing, sample enrichment, sequencing, target identification, mass spectrometry, or any combination thereof. The sequencing may be single-molecule sequencing, nanopore sequencing, fluorosequencing, or a combination thereof. The sequencing may be nucleic acid sequencing or peptide sequencing. The sequencing may comprise Edman degradation.

The method may further comprise isolating the peptide or protein from a biological sample. The biological sample may be derived from, for example, tissue, blood, urine, saliva, lymphatic fluid, or any combination thereof. The method may further comprise digesting the peptide or protein. The method may further comprise (i) isolating the peptide or protein from a biological sample, (ii) immobilizing the peptide or protein to a solid support, (iii) labeling at least one internal amino acid residue, and (iv) releasing the peptide or protein from said solid support. The immobilizing may comprise coupling a N-terminal amino acid residue of said peptide or protein to a capture moiety coupled to a solid support. The capture moiety may comprise an aldehyde, such as, for example, pyridine carboxaldehyde or a derivative thereof.

The peptide or protein may be a recombinant or a synthetic peptide or protein.

The protein or peptide may be reversibly modified by the reactive agent. The protein or peptide may be irreversibly modified by the reactive agent.

### Mass Spectrometry

The compositions and methods described herein may be useful for peptide and protein identification. The ability to add a functional group to a peptide C-terminal for improved mass spectrometry analysis (e.g., a bromine tag) may enable peptide quantification and identification. For example, techniques in C-terminal proteomics (e.g., the enrichment and identification of C-terminal peptides of digested proteins) can use such labeling strategies. Similar to isobaric tag methods implemented for labeling the N-termini of peptides (e.g., with cross reactivity to lysine residues), isobaric tags can be used to label the C-termini of peptides. The isobaric tags can be used for multiplexing protein samples from different samples as well as obtaining relative quantification of peptides, proteins, or combinations thereof in the different samples. The number of multiplexing in a sample can be doubled by tagging the N and the C terminal residues of a peptide or protein. Another improvement in peptide and protein identification by selectively labeling the C-terminus is for tandem mass spectrometry. The C-terminus of a protein or peptide can provide a highly charged group (e.g., positively charged amines, bromines, or negatively charged phosphates). Labeling the C-terminus of a peptide or protein may ensure substantially all the peptide fragments can ionize with equal efficiency, allowing more accurate protein and peptide identification.

### Sequencing

The compositions and methods described herein may be useful for peptide and protein sequencing.

### Nanopore Sequencing

Nanopore sequencing is a third-generation sequencing method of biopolymers, such as, for example polynucleotides. Both biological and solid-state methods exist. The method can utilize electrophoresis to transport a polymer through a small orifice, such as, for example, a porin protein, an unfoldase-protease pore complex, or nanometer sized holes in a metal or metal alloy. These small orifices can be embedded in a surface (e.g., a lipid membrane or metal or metal alloy), to create a porous surface. An electric current can be measured from the system, and the difference in electrical signal can be measured for each polymer subunit to determine the identity of that polymer subunit (e.g., DNA and RNA bases). In some cases, an amino acid or type of amino acid (e.g., all lysines in a peptide) may be coupled to a label that provides an identifiable electrical signal during pore transit. Alternatively or in combination with electric current measurement, translocation of the biopolymer through the pore may be monitored optically. For example, the pore may comprise a FRET donor configured to activate FRET acceptors on the biopolymer, such that translocation of the biopolymer through the pore may generate a time-resolvable FRET signal. A peptide may comprise a plurality of labels which each generate a signal upon translocation through the pore. A signal may identify an amino acid (e.g., identify the type of amino acid to which a label generating a signal is coupled) or a sequence (e.g., a sequence of three contiguous amino acids such as lysine-threonine-tyrosine) of the peptide. The system can be configured to quantify peptides or portions thereof (e.g., individual amino acids). A nanopore sequencing assay may identify a residue or a sequence of a peptide (e.g., a peptide coupled to a C-terminal coupling reagent). Considering the methods and compositions described herein, the biopolymers of nanopore sequencing may also be adapted as barcodes.

A C-terminal coupling reagent may comprise a detectable label (e.g., a handle comprising a detectable moiety such as a fluorophore), which may provide information in a nanopore sequencing assay. A detectable label may comprise a barcode (e.g., a nucleic acid or peptide barcode). The barcode may comprise information. For example, a sequence of a nucleic acid or peptide barcode may identify the sample or cell (e.g., a single cell from a cell sorting experiment or a cell from a colony) from which a C-terminal tagged peptide was derived. In some cases, a barcode sequence of a C-terminal coupling reagent is identified with nanopore sequencing. In some cases, a sequence of a nucleic acid barcode coupled to a peptide (e.g., by a C-terminal coupling reagent) and a sequence of the peptide are identified by nanopore sequencing. In some cases, a detectable label may be an optically detectable label, such as a fluorescent dye, a FRET donor or acceptor, or a quencher. In some cases, a detectable label may be an electrochemically detectable label (e.g., may comprise a characteristic oxidation or reduction potential).

The detectable label may generate a signal upon translocation through a pore. For example, an optically detectable label may generate a FRET signal upon transit past a pore-coupled FRET donor or acceptor, or an electrochemically detectable label may undergo detectable oxidation or reduction during transit through a pore. Detection of C-terminal transit through a pore can improve the accuracy of a nanopore sequencing method. For example, a nanopore sequencing method with detectably labeled peptide C-terminals can distinguish the beginning or end of pore translocation events, and thus distinguish two peptide translocations closely spaced in time. A nanopore sequencing method with detectably labeled peptide C-terminals may be able to identify the length of a peptide. For example, a method may comprise selectively labeling subject peptide C-termini with a first detectable label (e.g., coupling a C-terminal coupling reagent comprising a red dye) and N-termini (e.g., an amine or N-terminal specific label comprising a blue dye), such that the first and last position of a subject peptide may be identified during a pore translocation event.

The detectable label may also provide a detectable signal prior to or following transit through a pore. For example, a fluorescent label may enable quantification of tagged peptides prior and subsequent to translocation across a porous membrane, for example to enable quantitation of translocation efficiency.

A C-terminal coupling reagent may comprise a handle that affects pore translocation efficiency. A variety of nanopore sequencing methods drive pore or membrane translocation with an electrical potential that induces the movement of charged species (e.g., through a pore). While such techniques can be amenable to nucleic acids, which naturally bear net negative charges, electrical potential driven pore translocation of peptides is often more challenging, as peptides can contain positive, negative (e.g., aspartate residues), neutral (e.g., phenylalanine residues), and zwitterionic substituents (e.g., an ADP-ribosylated arginine). As such, among any plurality of peptides, only a subset will typically translocate through a pore or membrane in response to an electrical potential. The present disclosure provides compositions and methods for overcoming this limitation. In some cases, a C-terminal coupling reagent may comprise a charged label, such as a polyarginine or polyglutamate oligopeptide label. The positive or negative charge provided by such a label may enhance the efficiency or rate at which a C-terminal coupling reagent-coupled peptide translocates a pore or membrane in response to an electrical potential.

A C-terminal coupling reagent may also comprise an affinity for a pore or a species coupled to a pore. For example, A C-terminal coupling reagent may be coupled to a ligand which comprises a binding affinity for a pore protein, thereby localizing the C-terminal coupling reagent (and any peptide coupled thereto) to the pore, and increasing the likelihood of pore translocation by the peptide.

A method of the present disclosure may comprise coupling a C-terminal coupling reagent to a peptide and translocating the peptide through a pore (e.g., a nanopore), upon which translocating a signal is detected from the peptide, the C-terminal coupling reagent coupled thereto, or a combination thereof. The peptide may be derived from a virus, cell, or tissue sample (e.g., through lysis or homogenization). The peptide may be derived by cleaving another protein or peptide (e.g., chemically, such as with cyanogen bromide, or enzymatically, for example trypsinization). The C-terminal coupling reagent may comprise a detectable label. The detectable label may comprise a nucleotide or peptide sequence. The detectable label may comprise an optically or electrochemically detectable moiety. The C-terminal reagent may comprise a label that affects a pore translocation rate.

The signal may identify an amino acid of the peptide. The signal may identify at least a portion of the sequence of the peptide. The signal may identify a sequence of a barcode coupled to the C-terminal coupling reagent and at least a portion of the sequence of the peptide. The signal may comprise a plurality of distinct signals (e.g., a plurality of signals from a plurality of amino acid residues of the peptide). The method may comprise labeling an N-terminus or internal amino acid of said peptide, said label configured to provide said signal detected from said peptide during said translocating said peptide through said pore. The N-terminus or internal amino acid label may be an amino acid-type specific label. In such cases, said signal may identify said amino acid type. A peptide may comprise a plurality of N-terminal or internal amino acid labels. In some cases, a plurality of amino acids of a single type are labeled (e.g., all lysine residues in the peptide are labeled). In some cases, two or more types of amino acids are coupled to amino acid-type identifying labels (e.g., each lysine is labeled with a red dye and each cysteine is labeled with a green dye). A method may comprise labeling at least one, at least two, at least three, at least four, or at least five types of amino acids. An amino acid type-specific label may be configured to couple (e.g., to selectively couple) to lysine, cysteine, carboxylate side chain containing amino acids (e.g., aspartic acid and glutamic acid), tyrosine, tryptophan, arginine, histidine, serine, threonine, or any combination thereof. An amino acid type-specific label may be configured to couple to a non-natural or post-translationally modified amino acid, such as phosphotyrosine.

### Fluorosequencing

Fluorosequencing can provide single molecule resolution for the sequencing of proteins and peptides (Swaminathan, 2010; U.S. Pat. No. 9,625,469; U.S. patent application Ser. No. 15/461,034; U.S. patent application Ser. No. 15/510,962). One of the hallmarks of fluorosequencing is coupling of a fluorophore or other label to specific types of amino acid residues of a subject protein or peptide (e.g., the peptide to be fluorosequenced). This can involve labeling one or more amino acid residues with a labeling moiety. A fluorosequencing method may comprise labeling a single type of amino acid (e.g., every lysine or every cysteine) in a subject protein or peptide. A fluorosequencing method may comprise labeling a plurality of types of amino acid in a subject protein or peptide (e.g., lysine and tyrosine). A fluorosequencing method may comprise labeling one, two, three, four, five, six, or more different types of amino acids residues in a subject peptide or protein. The labeling moiety that may be used include, for example, fluorophores, chromophores, and quenchers. A plurality of amino acid residues may include, for example, an N-terminal amino acid, cysteine, lysine, glutamic acid, aspartic acid, tryptophan, tyrosine, serine, threonine, arginine, histidine, methionine, or any combination thereof. Each of these amino acid residues may be labeled with a different labeling moiety. Multiple amino acid residues may be labeled with the same labeling moiety such as aspartic acid and glutamic acid or asparagine and glutamine.

Labeling specificity is a major challenge in many fluorosequencing methods. In many cases, a label may comprise reactivity toward a plurality of amino acid types. For example, some maleimide labels can react with cysteine, lysine, and N-terminal amines. Discriminating between similarly reactive amino acid residues can require precise ordering of labeling steps. In the above maleimide example, lysine may be discriminated from cysteine by first reacting cysteine with a cysteine specific labeling step (e.g., iodoacetamide coupling at pH 7-8), thereby preventing further cysteine labeling in a subsequent lysine labeling step. A method may comprise cysteine labeling prior to lysine labeling. A method may comprise cysteine labeling prior to glutamate labeling. A method may comprise cysteine labeling prior to aspartate labeling. A method may comprise cysteine labeling prior to tryptophan labeling. A method may comprise cysteine labeling prior to tyrosine labeling. A method may comprise cysteine labeling prior to serine labeling. A method may comprise cysteine labeling prior to threonine labeling. A method may comprise cysteine labeling prior to histidine labeling. A method may comprise cysteine labeling prior to arginine labeling. A method may comprise lysine labeling prior to glutamate labeling. A method may comprise lysine labeling prior to aspartate labeling. A method may comprise lysine labeling prior to tryptophan labeling. A method may comprise lysine labeling prior to tyrosine labeling. A method may comprise lysine labeling prior to serine labeling. A method may comprise lysine labeling prior to threonine labeling. A method may comprise lysine labeling prior to arginine labeling. A method may comprise carboxylate side chain (e.g., glutamate and aspartate side chain) labeling prior to tryptophan labeling. A method may comprise carboxylate side chain (e.g., glutamate and aspartate side chain) labeling prior to tyrosine labeling. A method may comprise carboxylate side chain (e.g., glutamate and aspartate side chain) labeling prior to serine labeling. A method may comprise carboxylate side chain (e.g., glutamate and aspartate side chain) labeling prior to threonine labeling. A method may comprise carboxylate side chain (e.g., glutamate and aspartate side chain) labeling prior to histidine labeling. A method may comprise carboxylate side chain (e.g., glutamate and aspartate side chain) labeling prior to arginine labeling. A method may comprise at least 2, at least 3, at least 4, at least 5, or at least 6 amino acid labeling steps performed in a sequence configured to minimize or prevent label cross-reactivity (e.g., labeling more than the intended type or types of amino acids).

The present disclosure provides reagents, compositions, and methods for selectively labeling C-terminal carboxyl groups over carboxyl-containing amino acid side chains (e.g., aspartic acid and glutamic acid side chains). Differentially labeling a C-terminus (e.g., with a C-terminal capture reagent) and carboxyl-containing amino acid side chains in a peptide can enable multiple labeling steps prior to peptide immobilization (e.g., by a C-terminal capture reagent coupled to the C-terminus) or peptide analysis (e.g., fluorosequencing).

Accordingly, the present disclosure provides methods comprising (i) selectively coupling a reactive agent (e.g., a C-terminal coupling reagent) to a C-terminal carboxylate of a peptide and (ii) coupling a label to an N-terminal amino acid or to an internal amino acid of said peptide. In some cases, said selectively coupling said reactive agent to said C-terminal carboxylate of said peptide is subsequent to said coupling said label to said N-terminal amino acid or to said internal amino acid of said peptide. In some cases, said coupling said label to said N-terminal amino acid or to said internal amino acid of said peptide is subsequent to said selectively coupling said reactive agent to said C-terminal carboxylate of said peptide. Said label may be an amino acid type specific label, such as a lysine specific label, a cysteine specific label, a tyrosine specific label, a tryptophan specific label, a histidine specific label, a serine specific label, a threonine specific label, a specific label, an arginine specific label, a glutamic acid specific label, an aspartic acid specific label, an N-terminal amine specific label, or any combination thereof. In some cases, said label is a lysine specific label, a cysteine specific label, a glutamic acid specific label, an aspartic acid specific label, an N-terminal amine specific label, or any combination thereof.

A method may comprise quantifying peptides from a sample with a signal from a C-terminal coupling reagent. A method may comprise labeling the C-termini of peptides in a sample with C-terminal coupling reagents, removing (e.g., by washing) unreacted C-terminal coupling reagents, and quantifying the C-terminal coupling reagents present in the sample.

In some cases, the method comprises labeling a plurality of amino acids of said peptide (e.g., cysteine, lysine, and N-terminal amino acids). In such cases, said selectively coupling said reactive agent to said C-terminal carboxylate of said peptide may be subsequent to coupling a first label (e.g., an amino acid type specific label) to a first amino acid of said peptide and prior to coupling a second label (e.g., an amino acid type specific label with a different amino acid type specificity than the first label) to a second amino acid of said peptide. For example, a peptide labeling method may comprise labeling at least 1, at least 2, at least 3, at least 4, or at least 5 types of amino acids prior to selectively labeling a C-terminal carboxylate, and may further comprise labeling at least 1, at least 2, at least 3, at least 4, or at least 5 types of amino acids subsequently to said labeling of said C-terminal carboxylate.

While this technique may be used with labeling moieties, such as those described above, other labeling moieties may be used in fluorosequencing-like methods, such as synthetic oligonucleotides or peptide-nucleic acid. In particular, the labeling moiety used in the instant application may be suitable to withstand the conditions of removing one or more of the amino acid residues. Some non-limiting examples of potential labeling moieties that may be used in the instant methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5)6-napthofluorescein. The labeling moiety may be a fluorescent peptide or protein or a quantum dot.

Fluorosequencing may comprise removing peptides through techniques such as Edman degradation and subsequent visualization. Sequential peptide removal may generate sequence or position-specific information. For example, a reduction in fluorescence following an N-terminal amino acid removal step may indicate that a labeled amino acid, and thus that a specific type of amino acid, was disposed at a peptide N-terminal. Removal of each amino acid residue can carried out with a variety of different techniques including Edman degradation and proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide chain. In situations where Edman degradation is used, the amino acid residue at the N-terminus of the peptide chain is removed.

The methods of sequencing or imaging the peptide sequence may comprise immobilizing the peptide on a surface. The peptide may be immobilized to the surface by coupling a peptide-derived cysteine residue, the peptide N terminus, or the peptide C terminus with the surface or with a reagent coupled to the surface. The peptide may be immobilized by reacting the cysteine residue with the surface or with a capture reagent coupled to the surface. The peptide may be immobilized by coupling the peptide C-terminus with a C-terminal coupling reagent (e.g., a capture reagent comprising Formula (I)), and coupling the C-terminal coupling reagent to the surface or to a reagent coupled to the surface. The peptide may be immobilized on a surface. The surface may be optically transparent across the visible spectrum and/or the infrared spectrum. The surface may possesses a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 m thick, between 1 and 5 m thick, between 2 and 10 m thick, between 5 and 20 m thick, between 20 and 50 m thick, between 50 and 200 m thick, between 200 and 500 m thick, or greater than 500 m in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and modified targets for selection. Alternatively, an aminosilane modified surfaces may be used in the methods described herein. The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein involve imaging the peptide or protein to determine the presence of one or more labeling moieties (e.g., amino acid labels) coupled to the peptide. The sequencing technique may comprise imaging a plurality of peptides or proteins to determine the presence of one or more labeling moieties on individual peptides from among the plurality of peptides. The sequencing technique may comprise imaging at least 10³, at least 10⁴, at least 10⁵, at least 10⁶, at least 10⁷, at least 10⁸ or more proteins or peptides (e.g., imaging a portion of a surface comprising at least 10³ to at least 10⁸ proteins or peptides). These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (SEQ ID NO: 26, wherein ‘K’ denotes lysine, ‘D’ denotes aspartate, ‘Y’ denotes tyrosine, ‘A’ denotes alanine, and ‘G’ denotes glycine), and may comprise labels coupled to each lysine and tyrosine residue. A first image comprising the C-terminal immobilized peptide may indicate the presence of two lysines and one tyrosine in the peptide. The N-terminal amino acid may be removed (e.g., by Edman degradation), such that a second image comprising the C-terminal immobilized peptide may indicate the presence of one lysine and one tyrosine in the peptide. This process may be repeated until a sequence of KXXYXXXXXXXKX (SEQ ID NO: 27) is identified for the peptide, wherein ‘X’ indicates a non-lysine, non-tyrosine amino acid, ‘K’ indicates a lysine, and ‘Y’ indicates a tyrosine. A method of the present disclosure can identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and comparing these locations to known peptide sequences, which may identify the entire list of amino acid residues in the peptide sequence. For example, identifying the positions of the lysines and cysteines in a 40 amino acid fragment of a human protein may uniquely identify the protein (e.g., only one human protin contains the specific pattern of lysine and cysteine residues identified in the 40 amino acid fragment).

An imaging method may involve a variety of different spectrophotometric and microscopy methods, such as fluorimetry, diffuse reflectance, interferometric scattering, Raman, resonance enhanced Raman, infrared absorbance, visible light absorbance, ultraviolet absorbance, and fluorescence. The fluorescent methods may employ such fluorescent techniques, such as fluorescence polarization, Forster resonance energy transfer (FRET), or time-resolved fluorescence. A spectrophotometric or microscopy method may be used to determine the presence of one or more fluorophores coupled to a single peptide. Such imaging methods may be used to determine the presence or absence of a label on a specific peptide sequence. After repeated cycles of removing an amino acid residue and imaging a subject peptide, the position of the labeled amino acid residue can be determined in the peptide.

The length of a protein or peptide can be determined using the methods and compositions described herein. A C-terminal coupling reagent can comprise a barcode (e.g., a fluorophore or nucleic acid oligomer) that can be used to determine the length of the peptide molecule. Each cycle of degradation (e.g., Edman degradation) can be tallied; the sum total of the tally may correspond to the number of amino acids present in a peptide or protein. The removal of the fluorophore or the absence of a fluorescent hybridization event can indicate the number of amino acids present in a peptide or protein.

### C-Terminal Peptide Enrichment

Various aspects of the present disclosure provide methods for selectively functionalizing a peptide C-terminal with a reactive agent. The reactive agent may comprise a functional handle for purifying the peptide (e.g., biotin). The C-terminal amino acid of a protein or peptide may be the only amino acid in the protein that contains a functional handle. Protease digestion of proteins, peptides, or a combination thereof after labeling may generate peptide fragments that are not coupled to a reactive agent, and therefore do not contain a functional handle (e.g., biotin). For example, the C-terminus of a 20 amino acid peptide may be coupled to a C-terminal coupling reagent, and then cleaved at its 10^{th} amino acid, resulting in a first peptide fragment comprising the first ten amino acids of the original peptide and no C-terminal coupling reagent, and a second peptide fragment comprising the second ten amino acids of the original peptide comprising a C-terminus coupled to the reactive agent. Therefore, fragmentation (e.g., protease digestion) of a protein or peptide may generate a plurality of peptide fragments, wherein only a single peptide fragment of the plurality of peptide fragments is coupled to a reactive agent (and thereby a functional handle such as biotin).

A method may comprise selective peptide enrichment with a reactive agent functional handle (e.g., biotin). Such a method (e.g., streptavidin-based enrichment of biotin labeled peptides) may enrich a subpopulation of peptides from a complex mixture. The peptides, proteins, or a combination thereof can also be subjected to capture by a different functional handle that covalently immobilized peptide molecules for fluorosequencing. The methods and compositions described herein may provide improved analysis of a restricted number of proteins, peptides, or a combination thereof by increasing the relative quantification of the proteins, peptides, or combinations thereof in a sample. The stoichiometry of the proteins, peptides, or a combinations thereof in the sample may be improved by C-terminal labelling using selective handles.

### Multiplexing

A method of the present disclosure may comprise simultaneously analyzing a plurality of peptides derived from multiple, distinct samples (e.g., separate cell cultures or biopsy samples), wherein a peptide from the plurality of peptides may be labeled with a C-terminal coupling reagent comprising a handle (e.g., a nucleic acid barcode or a fluorophore) that identifies the sample from which the peptide was derived.

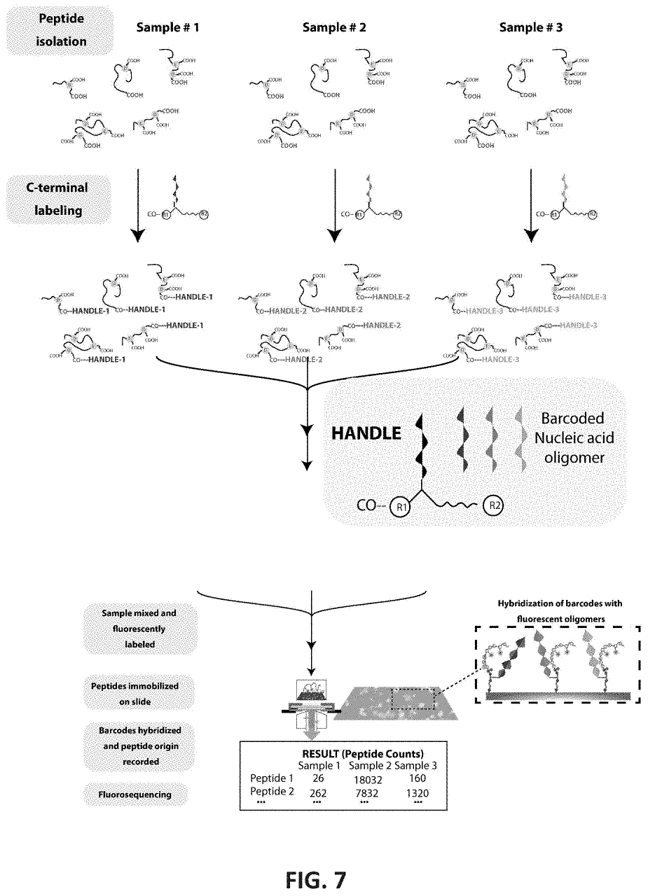

A schematic for peptide identification and quantification by multiplexing is shown in FIG. 7. The handle may comprise a nucleic acid oligomer (e.g., FIG. 6). The sequence of the nucleic acid oligomer may reflect the sample identity (e.g., a barcode). All peptides originating from a sample may contain the same sequence on the nucleic acid oligomer. The C-terminal ligation reaction on a different sample may comprise a unique barcode. The peptides, proteins, or a combination thereof may be mixed in the same reaction vials. The peptides, proteins, or a combination thereof may be labelled with, for example, fluorophores. After immobilization to a surface, a sequential or parallel flow of oligonucleotides that can hybridize with each of the known barcodes may be contacted to the peptides. The oligonucleotides may contain spectrally distinguishing fluorophores. The localization of the oligonucleotides can denote the sample identity for the peptide or protein. For example, a first sample may be contacted with a first reactive agent comprising a first barcode, a second sample may be contacted with a second reactive agent comprising a second barcode, and a third sample may be contacted with a third reactive agent comprising a third barcode. Subsequent to mixing (e.g., combining the first, second, and third samples post-reactive agent coupling), the sample of origin may be determined for each peptide through barcode identification. By ascribing sample identity to each peptide, protein, or combination thereof, the final analysis can indicate changes in quantitation as well as the ability to sequence a substantial number of samples. For example, protein expression may be simultaneously measured in a plurality of samples by contacting each sample with a reactive agent comprising a unique handle (e.g., a fluorophore with a distinguishing absorption or emission feature).

Selectively labeling the C-termini residue on peptides would be an important breakthrough for a number of high sensitivity analytical methods for studying proteomics. For example, selective terminal amino acid labeling could enable selective immobilization and differential labeling of peptides from complex mixtures. This could greatly enhance the utility of certain protein analytical methods, for example nanopore sequencing, which can provide accurate and reproducible protein detection and quantitation for a wide range of systems. Nanopore sequencing can provide a route for multiplexing proteins from different samples in the same nanopore experiment. Some of these newer methods are fluorosequencing, nanopore mediated protein sequencing or a number of peptide sequencing methods based upon N-terminal affinity reagents. This would be most likely given that the terminal recognition of peptides would result in selectivity for immobilization to solid surfaces or producing a differential charged end for translocating across pores.

### Sample Types

The methods described herein may comprise analyzing a biological sample. A biological sample may be derived from a subject (e.g., a patient or a participant in a study), from a tissue sample (e.g., an engineered tissue sample), from a cell culture (e.g., a human cell line or a bacterial colony), from a cell (e.g., a cell isolated during a single cell sorting assay), or a portion thereof (e.g., an organelle from a cell or an exosome from a blood sample). A biological sample may be synthetic, such as a composition of synthetic peptides. A sample may comprise a single species or a mixture of species. A biological sample may comprise biomaterial from a single organism, from a colony of genetically near-identical organisms, or from multiple organisms (e.g., enterocytes and microbiota from a human digestive tract). A biological sample may be fractionated (e.g., plasma separated from whole blood), filtered, or depleted (e.g., high abundance proteins such as albumin and ceruloplasmin removed from plasma).

A sample may comprise all or a subset of the biomolecules from the subject, tissue sample, cell culture, cell, or portion thereof. For example, a sample from a subject may comprise the majority of proteins present in that subject, or may comprise a small subset of the proteins from that subject. A biological sample may comprise a bodily fluid such as cerebral spinal fluid, saliva, urine, tears, blood, plasma, serum, breast aspirate, prostate fluid, seminal fluid, stool, amniotic fluid, intraocular fluid, mucous, or any combination thereof. A biological sample may comprise a tissue culture, for example a tumor sample, or tissue from a kidney, liver, lung, pancreas, stomach, intestine, bladder, ovary, testis, skin, colorectal, breast, brain, esophagus, placenta, or prostate.

The biological sample may comprise a molecule whose presence or absence may be measured or identified. The biological sample may comprise a macromolecule, such as, for example, a polypeptide or a protein. The macromolecule may be isolated (e.g., separated from other components from which it was sourced) or purified, such that the macromolecule comprises at least 0.5%, at least 1%, at least 2%, at least 3%, at least 4%, at least 5%, at least 7.5%, at least 10%, at least 15%, at least 20%, at least 25%, at least 30%, at least 40%, at least 50%, at least 60%, at least 70%, at least 75%, at least 80%, at least 90%, at least 95%, at least 98%, or at least 99% of a composition by weight (e.g., by dry weight or including solvent). The biological sample may be complex, and may comprise a plurality of components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single type (peptides, nucleic acids, lipids, small molecules). A biological sample may comprise a plurality of peptides configured for a method of the present disclosure (e.g., digestion, C-terminal labeling, or fluorosequencing).

Methods consistent with the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for different analyses (e.g., CTC lysate and buffycoat proteins may be partitioned and separately analyzed) or pooled for common analysis. A biological species may be homogenized, fragmented, or lysed prior to analysis. In particular instances, a species or plurality of species from among the homogenate, fragmentation products, or lysate may be collected for analysis. For example, a method may comprise collecting circulating tumor cells during a liquid biopsy, optionally isolating individual circulating tumor cells, lysing the circulating tumor cells, isolating peptides from the resulting lysate, and analyzing the peptides by a fluorosequencing method of the present disclosure. A method may comprise capturing peptides from a sample using a C-terminal capture reagent, and analyzing the peptides (e.g., by a fluorosequencing method).

Methods consistent with the present disclosure may comprise nucleic acid analysis, such as sequencing, southern blot, or epigenetic analysis. Nucleic acid analysis may be performed in parallel with a second analytical method, such as a fluorosequencing method of the present disclosure. The nucleic acid and the subject of the second analytical method may be derived from the same subject or the same sample. For example, a method may comprise collecting cell free DNA and a peptides from a human plasma sample, sequencing the cell free DNA (e.g., to identify a cancer marker), and performing proteomic analysis on the plasma proteins.

### EXAMPLES

### Example 1: Oxazolone Based Chemistry

This example provides a method for coupling a reactive agent to peptide C-termini and coupling handles to the C-termini-bound reactive agents, thereby yielding C-terminus labeled peptides. FIG. 3 provides an overview of C-terminal labeling method. Peptides (301, about 1 mg as a dry material) are solubilized in acetic anhydride and acetic acid (95:5 v/v) and are then incubated at 70° C. for 1 hour and dried in a speed vacuum yielding an oxazolone intermediate 302. Following re-suspension in H₂O/acetonitrile (50:50 v/v), HoBT and triethylamine (300 mM) are added, and the reaction mixture is allowed to incubate for ˜1 minute to hydrolyze anhydrides formed during the reaction. The resulting HOBt-derivatized peptides 303 are then combined with a reactive agent comprising a handle 304 at 50 mM, vortexed, and incubated for 4 hours at room temperature, yielding a reactive agent coupled to the C-terminus of a peptide 305. The peptides are provided for downstream analysis (e.g., sequencing). The peptides, proteins, or combinations thereof can be purified before or after downstream analysis. The handle may be configured for selective purification (e.g., the handle may comprise a Strep-tag for Streptactin-based purification).

### Example 2: Photoredox Chemistry

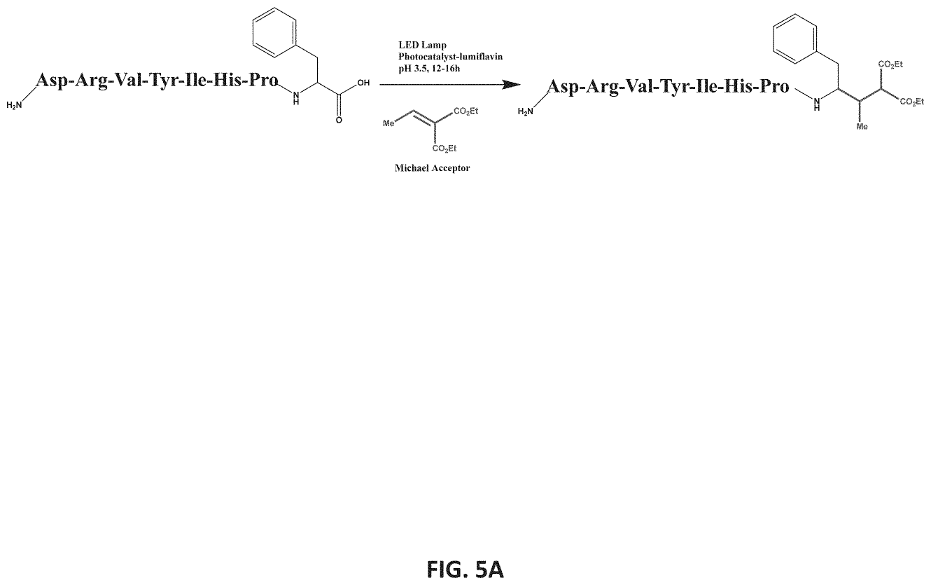

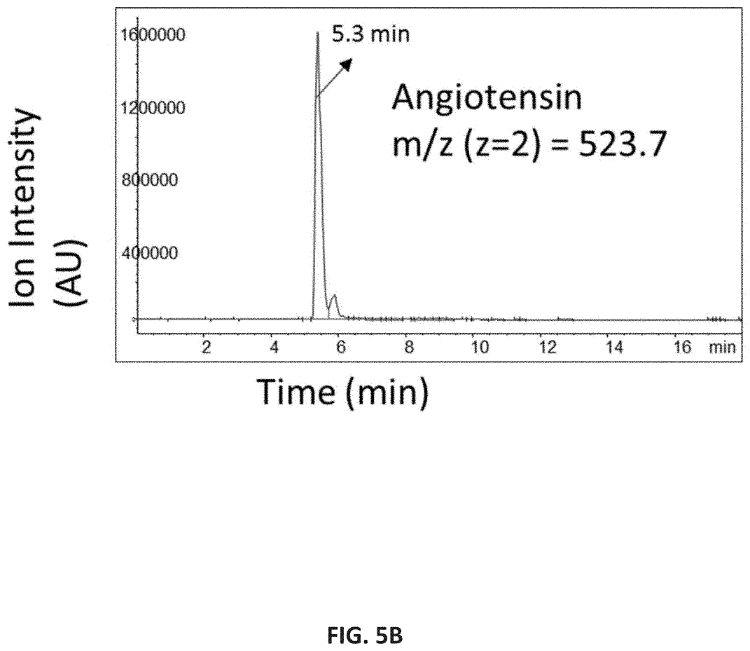

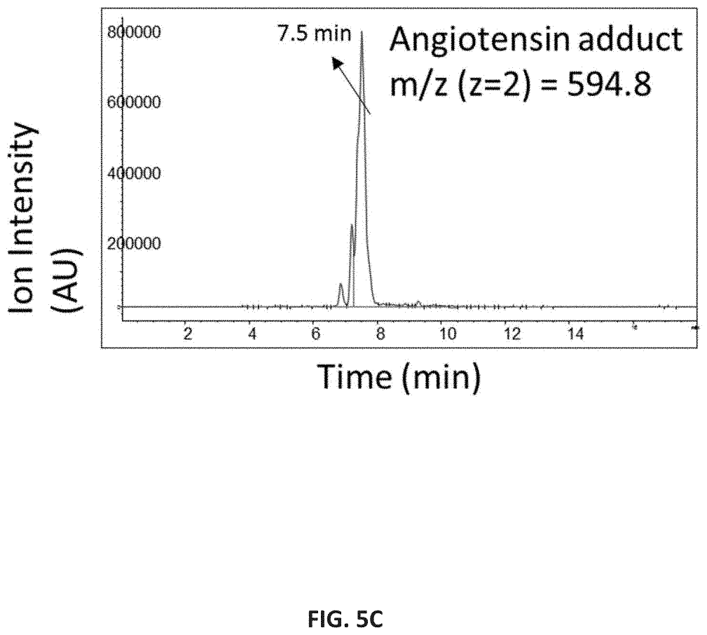

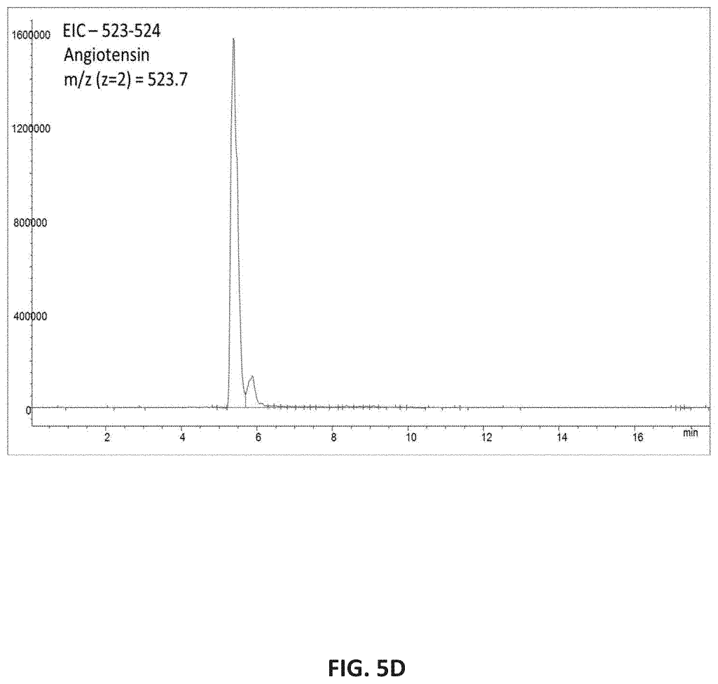

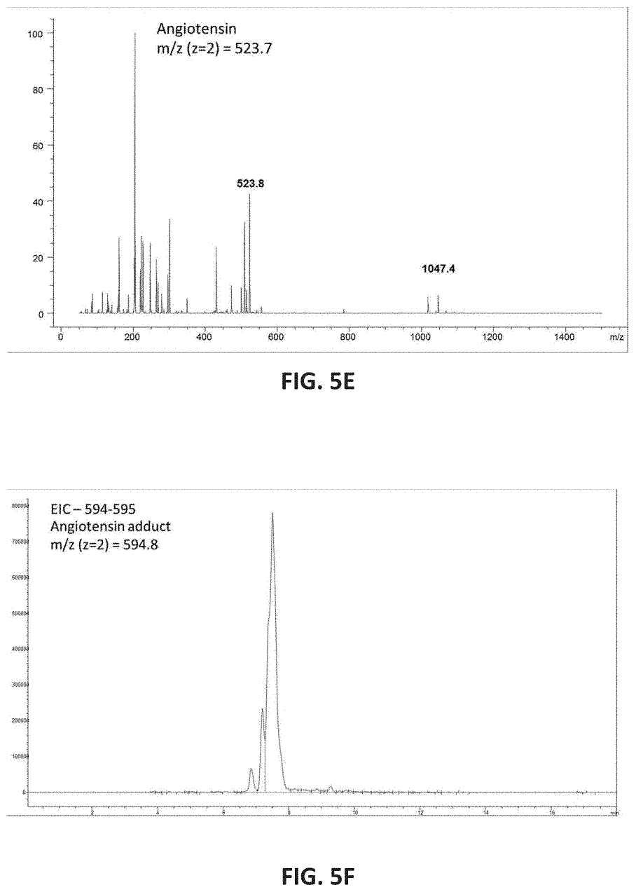

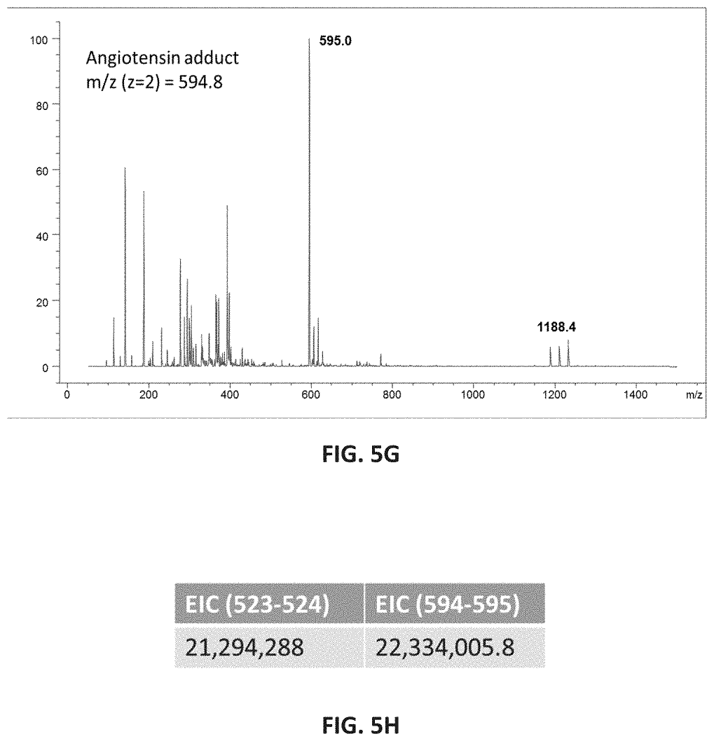

This example covers selectively reacting a peptide with a reactive agent comprising a Michael acceptor. In this example, the Michael acceptor is coupled directly to the peptide C-terminus without prior derivatization (e.g., conversion of the C-terminus to a reactive oxazolone prior to coupling to the reactive handle). As outlined in FIG. 5A, C-terminal specific labeling of Angiotensin II was performed with a lumiflavin photocatalyst and a full spectrum LED light source. A cooling system powered by a fan or other cooling source can be used. Lumiflavin is added at 30% mol/mol of the amount of the subject angiotensin fragment. In the example, diethyl ethylidenemalonate (e.g., 20 eq.) is used as the Michael acceptor configured to couple to the C-terminus of the Angeiotensin II peptides. Other Michael acceptors can be synthesized with terminal functional handles (e.g., alkynes or azides) or functional handles for barcoding (e.g., nucleic acid barcodes). Conversely, a functional handle may be appended to the reactive agent subsequent to C-terminal coupling (e.g., by nucleophilic substitution at an ethyl ester moiety of the reactive agent).

1 mg of Angiotensin-II is solubilized in 300 uL water and combined with 300 μL of 16.6% glycerol (e.g., making up to the total amount to 5% in 1 mL) and 100 μL of 0.1 M Sodium citrate buffer (pH 3.5). The resulting mixture is combined with buffer, glycerol, the lumiflavin photocatalyst, and the Michael acceptor (diethyl 2-ethylidenemalonate) in a 4-dram vial. The reaction is carried out for 12 h (overnight) under the LED light at room temperature. The total volume is made up to 1 mL. Nearly 40-50% of the Angiotenin II C-terminus is conjugated with the Michael acceptor. The LC-MS1 trace highlights the observed product in the crude final product (FIG. 5B-D).

### Example 3: Carboxypeptidase Ligation

The carboxylic acid group on peptides, proteins, or combinations thereof are esterified (e.g., alkyl ester (e.g., methyl ester), aryl ester, thioester) by incubating the dry peptide for 2 hours in 0.1M Methanolic HCl. The excess esterification reagent and water are removed, leaving behind a salt of the peptide, protein, or combination thereof. In other variants, the peptides, proteins, or combinations thereof are separated by dialysis with a 10 mM acetic acid in water as the buffer.

The esterified peptides, proteins, or a combination thereof are solubilized in about 50 μL of solubilization buffer (50 mM sodium acetate; 1% SDS at pH 5.5). In some cases, 1×PBS buffer (pH 7.2) is used to dissolve the peptides, proteins, or a combination thereof. In a prechilled microcentrifuge tube, 150 μL of sodium borate buffer (0.1M; pH 12.5) and 20 μL of 150 mM nucleophilic handle is added. Biocytinamide, which contains biotin at one end and amine being the reactive moiety, is used. 50 μL of the carboxypeptidase Y enzyme (0.1 mg/mL; ˜10 Units/mg) is added to the mixture along the sides. 150 μL of peptide-ester is added to the mixture and incubated for 30 minutes—2 hours at room temperature. The pH of the resulting solution is about 11.6. Increased incubation time removes the ester group from the peptide, protein, or combination thereof, and the transpeptidation reaction does not continue.

Carboxyamidomethyl (Cam) esters or substituted Cam esters (e.g., -Cam-Leu-OH and -Cam-Leu-NH₂) can be coupled to the C-termini of the donor peptide or protein. -Cam-Leu-NH₂ can be added with minimal self-esterification during the esterification of the donor peptide or protein. The Cam ester may be produced using Fmoc-Leu-rink amide resin.

The trans-peptidation reaction can be performed in solid or liquid phase. If liquid phase reaction is performed, the N-terminal peptide may be blocked with an electrophile (e.g., PCA). The functional group coupled to the C-terminus can be used to immobilize to the surface of a microscope slide.

### Example 4: Peptiligase Ligation

The Cam ester is washed multiple times and deprotected twice with 20% Piperidine in DMF at room temperature for 20 minutes. The resin is washed extensively with DMF. The carboxylic acid of glycolic acid (i.e., hydroxyacetic acid) is coupled to the amine on the resin through amide coupling chemistry (e.g., 1.5 eq of hydroxyacetic acid, 1.2 eq of HCTU, and 6 eq of DIPEA mixed with the deprotected Leu-rink amide resin for 3 h) prior to acid cleavage. It is cleaved with a TFA cocktail (e.g., 95% TFA, 2.5% H₂O and 2.5% triisopropyl silane) to release the HO-Cam-Leu-NH₂ molecule.

Peptide, proteins, or combinations thereof with protected amines are mixed with 5 eq of Leu substituted Cam alcohol, dissolved in dry DCM, and cooled to 0° C. In a separate vial, 1.2 eq of N-(3-Dimethylaminopropyl)-N′-ethylcarbodiimide hydrochloride (EDC) and 0.1 eq of 4-Dimethylaminopyridine (DMAP) are dissolved in dry DCM and cooled to 0° C. Under nitrogen, the two vials are mixed and stirred at room temperature for 3 hours. The end product is the conversion of all acidic groups on the donor peptide mixture to a Leu substituted Cam ester. The peptide is then solubilized in HEPES buffer (pH 8.0) for the Omniligase mediated ligation reaction.

75 μL of the esterified peptide (˜1 mg) is mixed with 2.5 μL of TCEP (100 mg/mL TCEP.HCl in water) and 25 μL of the nucleophilic handle. 2 μL of Ominiligase (10 U/mL) was added to the mixture and incubated for 2 h at room temperature. The esterified peptide ligates to the fixed linker (donor) molecule. The esterified aspartic and glutamic acid side chains are hydrolysed by elevating the pH to 12 with barium hydroxide.

### Example 5: C-Terminal Labeling with Norbornenone Reactive Agents

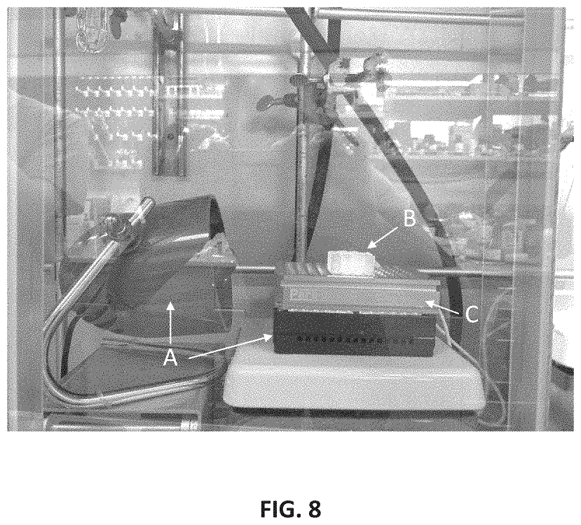

As another example, the C-terminal specific labeling procedure for peptide mixtures was optimized for coupling with the norbornenone variant using the principle of photoredox chemistry. The photoredox instrument—Lumidox II system (Analytical Sales and Services, New Jersey) fitted with the Blue LED (445 nm) at a power level of 110 mW and timed for 6 h incubation was setup. An active cooling base (Analytical Sales and Services, NJ) and a table fan was operated continuously to keep the contents cool. A photograph of the setup is shown in FIG. 8.

Reagents for the C-terminal reaction was are provided in three compositions—(a) a peptide mixture 901 (1nmole-1 μmole) solubilized in 100 uL buffer, such as water, phosphate buffer, acidic buffer, such as citrate etc, (b) photocatalyst mix—lumiflavine (0.1 mg/mL)—1-40% mol/mol of peptide) solubilized in 60 μL DMSO solvent (it can be substituted with water) and (c) 10 eq of a reactive agent comprising norbornenone 910—solubilized in 20 μL DMSO. The norbornenone-containing reactive agents used are—(i) norbornenone 910 and (ii) custom synthesized norbornenone-PEG4-Alkyne 911. The reaction mixture was made up to 500 μL with cesium formate buffer (pH 3.5).

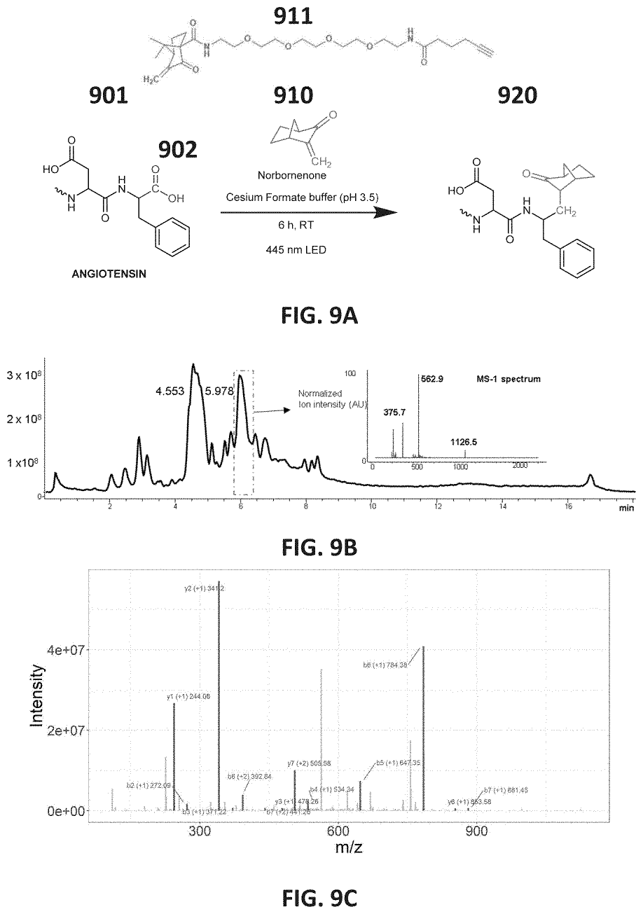

The reaction was first optimized with Angiotensin-II peptide and the LCMS trace indicating labeling of Angiotensin with the C-terminal norbornenone is shown in FIG. 9B. The high resolution tandem mass-spectrometry trace shown in FIG. 9C indicates that the norbornenone specifically reacts only at the C-terminal carboxylic acid and not the internal glutamic acid.

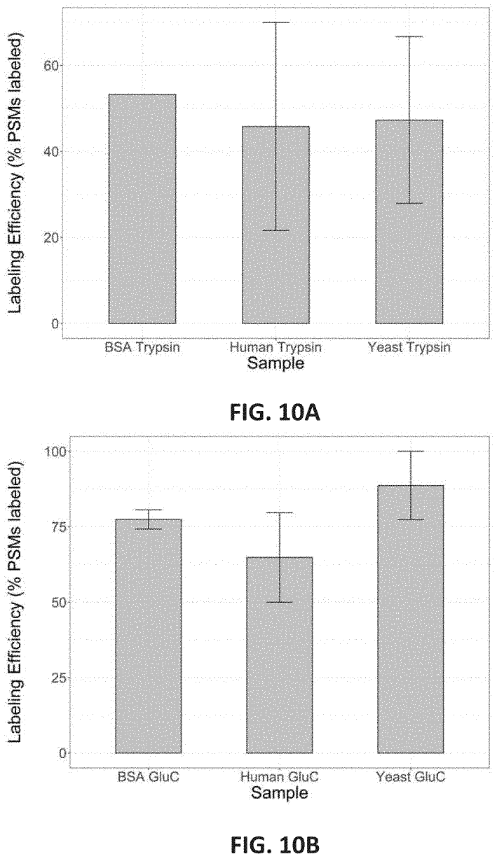

This method was repeated with more complex proteomic samples containing the tryptic digested peptides generated from 100 μg of bovine serum albumin (BSA), yeast and human protein isolates. The efficiency of labeling the C-termini was 65% on average FIG. 10A. An additional assay was performed on the gluC digestion products of the BSA, human protein, and yeast protein tryptic digestion products, resulting in increased C-terminal labeling efficiencies of nearly 90% FIG. 10B. Trypsin and gluC result in Lysine/Arginine and Aspartate/Glutamate as terminal residues respectively. This indicates the feasibility for use of this C-termini labeling chemistry with common proteomic proteases.

### Example 6: Effect of Terminal Amino Acid Type on Labeling Efficiency

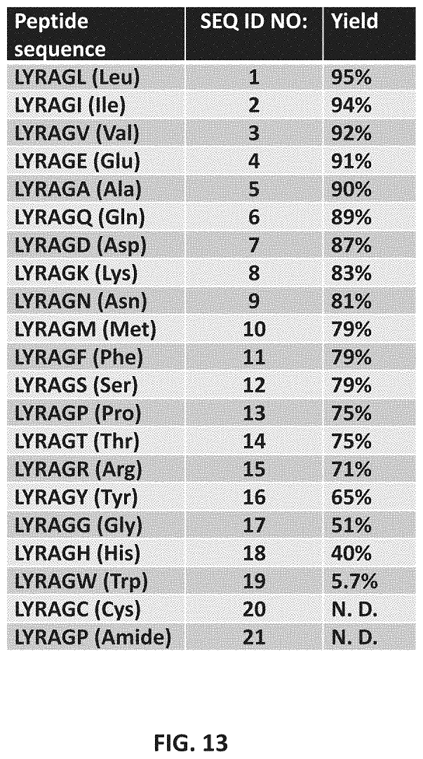

In order to understand if any terminal amino acid types bias labeling efficiency, we performed two orthogonal set of experiments. In the first class of experiments, 20 individual peptides each with a different C-terminal amino acid and comprising the sequence LYRAGX-OH (SEQ ID NO: 28, where ‘X’ represents any one of the 20 different canonical amino acids), was synthesized and assayed in triplicate for norbornenone coupling efficiency. As a negative control, we performed labeling with a C-terminal amide synthetic peptide LRWAG-ONH₂ (SEQ ID NO: 29), denoting a peptide comprising a C-terminal amide blocked from norbornenone labeling. The peptides peptide products were analyzed by LC-MS analytical instrument (Agilent) equipped with a 12 min 5-95% gradient of Water+0.1% Formic acid/Acetonitrile+0.1% Formic acid. As can be seen in FIG. 13, which summarizes the results of the assay, peptides with leucine C-termini provided the highest C-terminal labeling yield, while peptides with tryptophan, cysteine, and amide C-termini provided the lowest C-terminal labeling yield.

A second category of orthogonal experiments utilized the variability of terminal amino acids in peptides generated from proteins digested with proteases which cleave peptide bonds N-terminal of specific amino acid types. N-terminal specific proteases—AspN, LysN and Lysarginase and digested BSA protein, yeast and human protein isolate—were used to generate peptides with differing terminal amino acids. The extent of biases in labeling peptides based on their amino acids FIG. 11 were identified by analyzing the frequency of the terminal amino acids labeled and not-labeled with the norbornenone Michael acceptor. Variations were observed in labeling efficiency across experiments, which was sourced to the intrinsic challenge in separating and identifying the modified peptides in a complex sample with large background of photocatalyst and norbornenone. Commonly used purification steps such as C-18 tip cleanup or SP3 beads could not separate the photocatalyst from the peptides. It is conceivable that optimization of conditions, such as incubation times, % of DMSO in solutions, light intensity would further increase the labeling efficiency of the C-terminal adduct formation for proteomic applications.

### Example 7: Peptide Sequencing with Selective C-Terminal Labeling

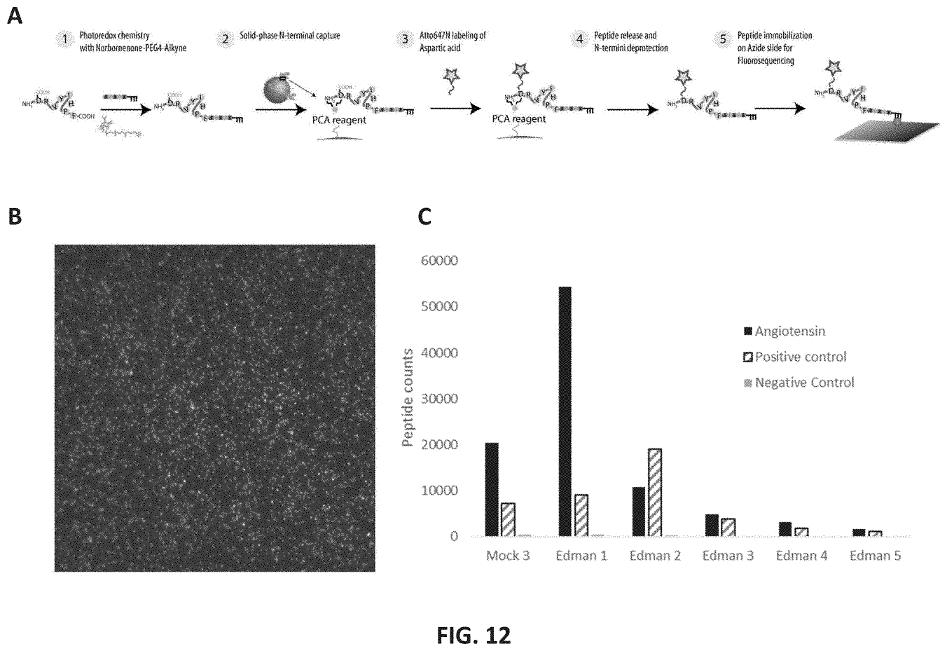

This example demonstrates a utility of the C-terminal selective labeling as a means for peptide immobilization in a fluorosequencing experiment. A series of labeling and substrate immobilization steps were performed as shown in FIG. 12 panel A, using Angiotensin, peptide-free water as a negative control, and a peptide of sequence AK*AGANY{PRA}R—ONH2 (SEQ ID NO: 24; *=Atto647N fluorophore; PRA=Propargylglycine) as a positive control in the fluorosequencing experiment. A Norbornenone-PEG4-Linker for use as the Michael acceptor. We performed a series of steps in the following order using Angiotensin as the positive control and water as the negative control prior to fluorosequencing. The steps are—FIG. 12 panel A(1) C-termini photo-redox chemistry to conjugate an alkyne moiety to the C-terminal end of the peptide (as described in Example 5); FIG. 12 panel A(2) Immobilization of the peptide on a first solid-phase support via the N-terminal amine; FIG. 12 panel A(3) labeling of internal acidic residues by HCTU/DIEA mediated amide coupling with Amine-Azide; FIG. 12 panel A(4) fluorescent Atto647N-PEG4-DBCO conjugation with copper-free click chemistry. The labeled peptides are cleaved from the resin and N-terminal deprotected, and then immobilized to a surface by the norbornenone-PEG4-Linker FIG. 12 panel A(5). Approximately 100,000 counts of fluorescent spots (comprising fluorescently labeled peptides and unreacted fluorophores) were sequenced using the fluorosequencing technology FIG. 12 panel B. The results of fluorosequencing are represented as the frequency of peptides losing fluorescent intensity after successive Edman degradative cycle FIG. 12 panel C.

These examples extend the use of the photoredox chemistry for selective and discriminative labeling of C-terminal carboxylic acid on peptides and other polymers. The method description and the demonstration will enable its broad utility across the different proteomic techniques.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-99. (canceled)

100. A method comprising:
(a) obtaining a peptide or protein, wherein said peptide or protein comprises a C-terminus comprising a first carboxylic acid moiety, and at least one internal amino acid, wherein said at least one internal amino acid is coupled to at least one label, wherein said at least one internal amino acid comprises a second carboxylic acid moiety; and
(b) coupling said first carboxylic acid moiety of said peptide or protein with a C-terminal coupling reagent preferentially over said second carboxylic acid moiety of said peptide or protein.

101. The method of claim 100, wherein coupling said first carboxylic acid moiety with said C-terminal coupling reagent is at least about 75% more preferential than coupling said second carboxylic acid moiety with said C-terminal coupling reagent.

102. The method of claim 100, wherein coupling said first carboxylic acid moiety with said C-terminal coupling reagent is at least about 90% more preferential than coupling said second carboxylic acid moiety with said C-terminal coupling reagent.

103. The method of claim 100, wherein said peptide or protein comprises at least two internal amino acids, wherein at least one of said at least two internal amino acids comprises said second carboxylic acid moiety.

104. The method of claim 100, wherein said C-terminal coupling reagent comprises a nucleophile or an electrophile.

105. The method of claim 104, wherein said nucleophile comprises an amine, an alcohol, a sulfide, a thiol, a cyanate, a thiocyanate, or any combination thereof.

106. The method of claim 104, wherein said electrophile comprises a Michael acceptor, an alkene, a diene, an acrylamide, an N-(prop-2-yn-1-yl)methylacrylamide, an isocyanate, an isothiocyanate, an oxirane, α,β-unsaturated carbonyl, a vinyl sulfone, or any combination thereof.

107. The method of claim 106, wherein said electrophile comprises said Michael acceptor.

108. The method of claim 107, wherein said Michael acceptor comprises 3-methylene-2-norbornanone or a derivative thereof.

109. The method of claim 100, wherein said C-terminal coupling reagent comprises a functionalization moiety.

110. The method of claim 109, wherein said functionalization moiety comprises an alkyne, an azide, a fluorophore, biotin, a nucleic acid molecule, an amino acid, a peptide, a solid support bead or resin, or any combination thereof.

111. The method of claim 100, wherein said C-terminal coupling reagent does not substantially couple to (i) said at least one internal amino acid and (ii) an N-terminal amino acid of said peptide or protein.

112. The method of claim 111, wherein said at least one internal amino acid, said N-terminal amino acid of said peptide or protein, or a combination thereof, is reversibly modified.

113. The method of claim 112, wherein said at least one internal amino acid, said N-terminal amino acid of said peptide or protein, or a combination thereof, is modified prior to coupling said C-terminal coupling reagent to said first carboxylic acid moiety.

114. The method of claim 112, wherein said at least one internal amino acid, said N-terminal amino acid of said peptide or protein, or a combination thereof, is modified subsequent to coupling said C-terminal coupling reagent to said first carboxylic acid moiety.

115. The method of claim 100, wherein said at least one label comprises an amino acid type-specific label.

116. The method of claim 100, wherein said at least one label comprises an optical label.

117. The method of claim 116, wherein said optical label comprises a fluorophore.

118. The method of claim 100, further comprising isolating said peptide or protein from a biological sample.

119. The method of claim 118, wherein said biological sample is derived from tissue, blood, urine, saliva, lymphatic fluid, or any combination thereof.


SEQUENCE LISTING (29 sequences)

SEQ ID NO: 1 - protein, 6 aa, synthetic construct
  REGION 1..6: note = Synthetic peptide
LYRAGL

SEQ ID NO: 2 - protein, 6 aa, synthetic construct
  REGION 1..6: note = Synthetic peptide
LYRAGI

SEQ ID NO: 3 - protein, 6 aa, synthetic construct
  REGION 1..6: note = Synthetic peptide
LYRAGV

SEQ ID NO: 4 - protein, 6 aa, synthetic construct
  REGION 1..6: note = Synthetic peptide
LYRAGE

SEQ ID NO: 5 - protein, 6 aa, synthetic construct
  REGION 1..6: note = Synthetic peptide
LYRAGA

SEQ ID NO: 6 - protein, 6 aa, synthetic construct
  REGION 1..6: note = Synthetic peptide
LYRAGQ

SEQ ID NO: 7 - protein, 6 aa, synthetic construct
  REGION 1..6: note = Synthetic peptide
LYRAGD

SEQ ID NO: 8 - protein, 6 aa, synthetic construct
  REGION 1..6: note = Synthetic peptide
LYRAGK

SEQ ID NO: 9 - protein, 6 aa, synthetic construct
  REGION 1..6: note = Synthetic peptide
LYRAGN

SEQ ID NO: 10 - protein, 6 aa, synthetic construct
  REGION 1..6: note = Synthetic peptide
LYRAGM

SEQ ID NO: 11 - protein, 6 aa, synthetic construct
  REGION 1..6: note = Synthetic peptide
LYRAGF

SEQ ID NO: 12 - protein, 6 aa, synthetic construct
  REGION 1..6: note = Synthetic peptide
LYRAGS

SEQ ID NO: 13 - protein, 6 aa, synthetic construct
  REGION 1..6: note = Synthetic peptide
LYRAGP

SEQ ID NO: 14 - protein, 6 aa, synthetic construct
  REGION 1..6: note = Synthetic peptide
LYRAGT

SEQ ID NO: 15 - protein, 6 aa, synthetic construct
  REGION 1..6: note = Synthetic peptide
LYRAGR

SEQ ID NO: 16 - protein, 6 aa, synthetic construct
  REGION 1..6: note = Synthetic peptide
LYRAGY

SEQ ID NO: 17 - protein, 6 aa, synthetic construct
  REGION 1..6: note = Synthetic peptide
LYRAGG

SEQ ID NO: 18 - protein, 6 aa, synthetic construct
  REGION 1..6: note = Synthetic peptide
LYRAGH

SEQ ID NO: 19 - protein, 6 aa, synthetic construct
  REGION 1..6: note = Synthetic peptide
LYRAGW

SEQ ID NO: 20 - protein, 6 aa, synthetic construct
  REGION 1..6: note = Synthetic peptide
LYRAGC

SEQ ID NO: 21 - protein, 6 aa, synthetic construct
  REGION 1..6: note = Synthetic peptide
LYRAGP

SEQ ID NO: 22 - protein, 10 aa, synthetic construct
  REGION 1..10: note = Synthetic peptide
ELYAEKVATR

SEQ ID NO: 23 - protein, 7 aa, synthetic construct
  REGION 1..7: note = Synthetic peptide
DRVYIHP

SEQ ID NO: 24 - protein, 8 aa, synthetic construct
  REGION 1..8: note = Synthetic peptide
AKAGANYR

SEQ ID NO: 25 - skipped

SEQ ID NO: 26 - protein, 14 aa, synthetic construct
  REGION 1..14: note = Synthetic peptide
KDDYAGGGAAGKDA

SEQ ID NO: 27 - skipped

SEQ ID NO: 28 - protein, 6 aa, synthetic construct
  REGION 1..6: note = Synthetic peptide
  SITE 6: note = misc_feature - Xaa can be any naturally occurring amino acid
LYRAGX

SEQ ID NO: 29 - protein, 5 aa, synthetic construct
  REGION 1..5: note = Synthetic peptide
LRWAG